(19) United States
(12) Patent Application Publication
(10) Pub. No.: US 2019/0241955 A1
(43) Pub. Date: Aug. 8, 2019

(54) METHODS OF DETERMINING ENDOMETRIAL RECEPTIVITY AND USES THEREOF

(71) Applicant: CooperSurgical, Inc., Trumbull, CT (US)

(72) Inventors: Katharina Späth, Oxford (GB); Dagan Wells, Oxford (GB)

(21) Appl. No.: 16/265,398

(22) Filed: Feb. 1, 2019

Related U.S. Application Data
(60) Provisional application No. 62/625,462, filed on Feb. 2, 2018.

(51) Int. Cl. C12Q 1/6876; A61B 17/435

(52) U.S. Cl. CPC C12Q 1/6876; C12Q 2600/158; A61B 17/435

(57) ABSTRACT

Provided herein are methods and kits for determining endometrial receptivity status.

### CLAIM OF PRIORITY

This application claims the benefit of U.S. Provisional Patent Application No. 62/625,462, filed on Feb. 2, 2018. The entire contents of the foregoing are hereby incorporated by reference.

### TECHNICAL FIELD

The present disclosure relates generally to the field of reproductive medicine. More specifically, this disclosure relates to in vitro methods and kits for determining endometrial receptivity status.

### BACKGROUND

The endometrium reaches a receptive status for embryonic implantation around day 19-21 of the menstrual cycle. The number of molecular diagnostic tools available to characterize the receptive status for embryonic implantation is very limited and lack key elements for the accurate determination of the window of implantation (WOI), such as immune response genes, crucial for embryo implantation.

### SUMMARY

In one aspect, the disclosure provides methods of predicting endometrial receptivity of a human subject for transplantation of a preimplantation embryo.

Provided herein are methods of predicting endometrial receptivity for embryonic implantation in a human subject that include: (a) providing a first biological sample obtained from a human subject at a first time point within a menstrual cycle; (b) determining the gene expression profile of a panel of genes in the first biological sample, wherein the panel of genes comprises two or more genes selected from the group consisting of: Annexin A4 (ANXA4), Apolipoprotein D (APOD), Apolipoprotein L2 (APOL2), Aquaporin-3 (AQP3), Amphiregulin (AREG), Arginase-2 (ARG2), ATP synthase F1 subunit beta (ATP5F1B), Calpain-6 (CAPN6), Corepressor interacting with RBPJ (CIR1), CKLF-like MARVEL transmembrane domain-containing protein 5 (CMTM5), Catenin alpha 2 (CTNNA2), C-X-C motif chemokine ligand 1 (CXCL1), C-X-C motif chemokine ligand 6 (CXCL6), Estrogen receptor 1 (ESR1), Forkhead box P3 (FOXP3), Growth arrest and DNA damage-inducible alpha (GADD45A), Gap junction protein alpha 4 (GJA4), Glutathione peroxidase 3 (GPX3), Hemoglobin subunit alpha (HBA1), Hemoglobin subunit gamma-1 (HBG1), Hydroxymethylbilane synthase (HMBS), Homeobox B7 (HOXB7), Insulin-like growth factor-binding protein 1 (IGFBP1), Interleukin-1 Receptor type 1 (IL1R1), Interleukin-4 (IL4), Interleukin-5 (IL5), Integrin subunit alpha-9 (ITGA9), Integrin beta-1 (ITGB1), Monoamine oxidase A (MAOA), Mitogen-activated protein kinase 1 (MAPK1), Matrix Metallopeptidase 9 (MMP9), Metallothionein-1E (MT1E), Metallothionein-1F (MT1F), Metallothionein-1H (MT1H), Metallothionein-1L (MT1L), Metallothionein-1X (MT1X), NF-kappa-B inhibitor alpha (NFKBIA), Nicotinamide N-methyltransferase (NMMT), Progesterone Receptor (PGR), progesterone receptor membrane component 1 (PGRMC1), phospholipase A2 group IVA (PLA2G4A), Prostaglandin-endoperoxidase synthase 1 (PTGS1), Ras homolog family member A (RHOA), ribosomal protein L13a (RPL13A), Secretoglobin family 2A member 2 (SCGB2A2), Succinate dehydrogenase complex flavoprotein subunit A (SDHA), Serpin family A member 1 (SERPINA1), and Trefoil factor 3 (TFF3) using quantitative real-time polymerase chain reaction (real-time qPCR) analysis; and (c) identifying the human subject as having: (i) a receptive endometrial status, when the determined gene expression profile corresponds to a gene expression profile of the panel of genes of a receptive endometrial receptivity reference group, (ii) a non-receptive endometrial status, when the determined gene expression profile corresponds to a gene expression profile of the panel of genes of a non-receptive endometrial receptivity reference group, (iii) a pre-receptive endometrial status, when the determined gene expression profile corresponds to a gene expression profile of the panel of genes of a pre-receptive endometrial receptivity reference group, or (iv) a post-receptive endometrial status, when the determined gene expression profile corresponds to a gene expression profile of the panel of genes of a post-receptive endometrial receptivity reference group.

In some embodiments, the first biological sample is an endometrial biopsy sample obtained from the uterine fundus.

In some embodiments, the human subject has previously undergone assisted reproductive treatment, and the first time point is seven days after a luteinizing hormone surge.

In some embodiments, the human subject has previously undergone assisted reproductive treatment, and the first time point is seven days after administration of human chorionic gonadotropin (hCG).

In some embodiments, the human subject has previously undergone hormone replacement therapy cycles, and the first time point is five days after progesterone administration.

In some embodiments of any of the methods described herein, the method further includes after identifying the human subject as having a receptive endometrial status, (d) transferring a pre-implantation embryo into the identified human subject.

In some embodiments of any of the methods described herein, the method further includes after identifying the human subject as having a non-receptive endometrial status, a pre-receptive endometrial status, or a post-receptive endometrial status, (d) obtaining a second biological sample from the human subject at a second time point and repeating steps (b) and (c) on the second biological sample.

In some embodiments of any of the methods described herein, the method further includes after identifying the human subject as having a receptive endometrial status, (e) transferring a pre-implantation embryo into the identified human subject.

In some embodiments, the second biological sample is an endometrial biopsy obtained from the uterine fundus.

In some embodiments, the human subject is identified as having a post-receptive endometrial status, and the second biological sample is obtained from the human subject in another menstrual cycle, one or two days before the first biological sample was obtained from the human subject in the previous menstrual cycle.

In some embodiments, the human subject is identified as having a pre-receptive endometrial status, and the second biological sample is obtained from the human subject in another menstrual cycle, one or two days after the first biological sample was obtained from the human subject, in the previous menstrual cycle.

In some embodiments wherein the human subject is identified as having a non-receptive endometrial status, the method further includes instructing a healthcare professional to select a treatment plan for the identified human subject.

In some embodiments wherein the human subject is identified as having a non-receptive endometrial status, the method further includes selecting a treatment plan for the identified human subject.

In some embodiments, the treatment plan includes a hormone replacement therapy cycle.

In some embodiments, the human subject has a history of miscarriage(s) or stillbirth(s), and/or a history of fertility issues.

In some embodiments, the human subject has had one or more cycles of in vitro fertilization (IVF).

In some embodiments, the human subject has previously not had IVF.

In some embodiments, the determining step occurs on a chip, an array, a multi-well plate, or a tube (e.g., a microcentrifuge tube).

In some embodiments, the determining step occurs in an array including through-holes pre-spotted with primer and probe pairs.

In some embodiments, the determining step of each gene within the panel of genes is performed in a reaction volume of 0.005 μL to 100 μL.

In some embodiments, the determining step of each gene within the panel of genes is performed in a reaction volume of 0.02 μL to 0.05 μL.

In some embodiments, the determining step is performed using a computer-assisted algorithm.

In some embodiments of any of the methods described herein, the method further includes modifying the human subject's clinical record to identify the human subject as having or not having a receptive endometrial status, as having or not having a post-receptive endometrial status, as having or not having a pre-receptive endometrial status, or as having or not having a non-receptive endometrial status. The clinical record may be stored in any suitable data storage medium (e.g., a computer readable medium).

Also provided herein are kits that include reagents suitable for determining an endometrial gene expression profile of a panel of genes in a biological sample obtained from a human subject, wherein the panel of genes comprises two or more genes selected from the group consisting of: ANXA4, APOD, APOL2, AQP3, AREG, ARG2, ATP5F1B, CAPN6, CIR1, CMTM5, CTNNA2, CXCL1, CXCL6, ESR1, FOXP3, GADD45A, GJA4, GPX3, HBA1, HBG1, HMBS, HOXB7, IGFBP1, IL1R1, IL4, IL5, ITGA9, ITGB1, MAOA, MAPK1, MMP9, MT1E, MT1F, MT1H, MT1L, MT1X, NFKBIA, NMMT, PGR, PGRMC1, PLA2G4A, PTGS1, RHOA, RPL13A, SCGB2A2, SDHA, SERPINA1 and TFF3.

In some embodiments, the biological sample is an endometrial biopsy obtained from the uterine fundus.

In some embodiments, the reagents are suitable for performing real-time qPCR.

In some embodiments of any of the kits described herein, the kit can further include reagents suitable for performing reverse transcription and multiplex pre-amplification.

In some embodiments of any of the kits described herein, the kit can further include a chip, an array, a multi-well plate or a tube (e.g., a microcentrifuge tube). In some embodiments, the array includes through-holes pre-spotted with primer and probe pairs.

In some embodiments of any of the kits described herein, the kit can further include instructions for use of the kit according to any of the methods described herein.

Also provided in aspects of the invention are panels of genes useful for predicting endometrial receptivity for embryonic implantation in a human subject. By “a panel of genes” it is meant a collection, or combination, of two or more genes, e.g., two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-five, sixty, sixty-five, seventy, seventy-five, eighty, eighty-five, ninety, ninety-five, one hundred, one hundred and ten, one hundred and twenty, one hundred and thirty, one hundred and forty, one hundred and fifty, one hundred and sixty, one hundred and seventy, one hundred and eighty, one hundred and ninety, two hundred genes (or any of the subranges of this range described herein), whose gene expression profile in a biological sample (e.g., an endometrial tissue biopsy sample) is associated with endometrial receptivity. The panel of genes (e.g., panel A) described herein may be used to provide a prediction of endometrial receptivity of a human subject, to monitor a subject with fertility issues (e.g., recurrent miscarriages, recurrent failed cycles of in vitro fertilization (IVF)), to monitor a subject undergoing IVF, to provide a prognosis to a human subject having a receptive endometrial status, to provide a prognosis to a human subject having a non-receptive endometrial status, to provide a prognosis to a human subject having a pre-receptive endometrial status, to provide a prognosis to a human subject having a post-receptive endometrial status.

In some embodiments, the methods include instructing a healthcare professional (e.g., a physician, physician assistant, nurse practitioner, nurse and case manager) to select a treatment plan for a subject. For example, the methods may further include selecting a treatment plan for a subject, which includes selectively administering a hormone replacement therapy (e.g., progesterone and estrogen), and/or performing a fertility evaluation, which includes, for example, performing a procedure selected from the group consisting of: an ultrasound, a hysterosalpingogram, a hysteroscopy and a hormone blood test. The treatment plan can include prescribing to the subject therapeutic lifestyle changes to improve fertility (e.g., dietary changes, weight loss or weight gain)

As used herein, the term “biological sample” refers to a sample obtained or derived from a subject. By way of example, the sample may be selected from the group consisting of body fluids (e.g., blood, whole blood, plasma, serum, mucus secretions, urine, or saliva) and tissue (e.g., an endometrial tissue biopsy sample). In some embodiments, the sample is, or includes a blood sample. The preferred biological source is an endometrial tissue biopsy sample.

The term “subject” as used herein refers to a mammal. A subject therefore refers to, for example, dogs, cats, horses, cows, pigs, guinea pigs, humans and the like. When the subject is a human, the subject may be referred to herein as a patient. The human subject can be genetically female (having XX sex chromosomes, or XXY sex chromosomes), premenopausal, and/or of advanced maternal age (e.g., over 35 years of age). For example, the human subject can have a history of miscarriages or stillbirths, a history of fertility issues/complications (e.g., pelvic inflammatory disease, endometriosis, polycystic ovarian syndrome, hormonal imbalances, premature ovarian aging/failure, antiphospholipid syndrome), and/or has previously had assisted reproductive treatments and/or hormone replacement therapies. In some examples, the human subject has had one or more cycles of IVF. In other examples, the subject has not had IVF.

As used herein, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). Directly obtaining a sample includes performing a process that includes a physical change in a physical substance, e.g., a starting material, such as a tissue biopsy, (e.g., endometrial biopsy that was previously isolated from a patient). Thus, obtain is used to mean collection and/or removal of the sample from the subject.

As used herein the term “reference group” refers to a group of endometrial tissue biopsy samples that are obtained from a group of individuals for which the endometrial status is known. A “receptive endometrial reference group” means that the gene expression profile of the selected genes (e.g., panel A) is for a group of individuals with a receptive endometrial status. A “non-receptive endometrial reference group” means that the gene expression profile of the selected genes (e.g., panel A) is for a group of individuals with non-receptive endometrial status. A “pre-receptive endometrial reference group” means that the gene expression profile of the selected genes (e.g., panel A) is for a group of individuals with pre-receptive endometrial status. A “post-receptive endometrial reference group” means that the gene expression profile of the selected genes (e.g., panel A) is for a group of individuals with a post-receptive endometrial status.

As used herein, the phrase “receptive endometrial status” means that the window of implantation matches the day on which the biological sample (e.g., biopsy sample) was taken, and that the subject's uterus is receptive for embryonic implantation. For example, a receptive endometrial status is defined as an endometrial status that is observed 7 days after the luteinizing hormone (LH) surge in a natural menstrual cycle of 28 days. In another example, a receptive endometrial status is defined as an endometrial status that is observed 6-10 days after the LH surge.

The phrase “pre-receptive endometrial status” means that the window of implantation has not yet been reached on the day on which the biological sample (e.g., biopsy sample) was taken, and that the subject's uterus is not receptive for embryonic implantation. A pre-receptive endometrial status is defined as an endometrial status that is observed in the days prior to the window of implantation during the secretory phase of a menstrual cycle.

The phrase “post-receptive endometrial status” means that the window of implantation has already passed on the day on which the biological sample (e.g., biopsy sample) was taken, and that the subject's uterus is not receptive for embryonic implantation. A post-receptive endometrial status is defined as an endometrial status that is observed in the days following the window of implantation during the secretory phase of a menstrual cycle.

The phrase “non-receptive endometrial status” means that the day on which the biological sample (e.g., biopsy sample) was taken was during the proliferative phase of a menstrual cycle, and that the subject's uterus is not receptive for embryonic implantation.

A “luteinizing hormone (LH) surge” can be determined using various methods known in the art, including by urine and/or blood testing, in order to detect the expression and/or presence of LH in a sample, and/or to quantify the amount of LH present in a sample. Expression and/or presence of LH can be detected using known assays that include antibodies targeting LH. Kits for determining a LH surge are commercially available and known to those in the art (e.g., the enzyme-linked immunosorbent assays (ELISAs) commercially available from Abcam and Invitrogen).

The phrase “the endometrial gene expression profile corresponds to an endometrial gene expression profile of the panel of genes of a reference group” means that the endometrial gene expression profile of a sample is predicted based on computer-assisted algorithms (e.g., principle component analysis, or any other classification algorithms known in the art) to fall within the classification of the endometrial gene expression profile of a reference group (e.g., a receptive endometrial reference group, a non-receptive endometrial reference group, a pre-receptive endometrial reference group, a post-receptive endometrial reference group).

As used herein, the phrase “assisted reproductive treatment (ART)” refers to a plurality of treatments that may facilitate fertility treatment. Non-limiting examples of ART include in vitro fertilization (IVF), gamete intrafallopian transfer (GIFT), zygote intrafallopian transfer (ZIFT), surrogacy, pre-implantation genetic testing, in vitro oocyte maturation, and hormone replacement therapy (HRT) cycles (e.g., exogenous administration of progesterone and estrogen).

As used herein the term “subfertile” refers to a subject who has difficulty getting pregnant or carrying a pregnancy to full-term. For example, a subject may be subfertile because of endometriosis, ovulatory disorders, tubal disease, peritoneal adhesion, and/or uterine abnormalities. For example, a subject may be subfertile if the subject is of advanced age (i.e. over 35 years of age).

As used herein the term “pre-implantation embryo” refers to a period of time that exists post-fertilization (in vitro or in vivo) and prior to the attachment of the embryo to the endometrium. In some embodiments, the period of time that exists post-fertilization (in vitro or in vivo) and prior to the attachment of the embryo to the endometrium is about 1 day to about 7 days (e.g., about 1 day to about 6 days, about 1 day to about 5 days, about 1 day to about 4 days, about 1 day to about 3 days, about 1 day to about 2 days, about 2 days to about 7 days, about 2 days to about 6 days, about 2 days to about 5 days, about 2 days to about 4 days, about 2 days to about 3 days, about 3 days to about 7 days, about 3 days to about 6 days, about 3 days to about 5 days, about 3 days to about 4 days, about 4 days to about 7 days, about 4 days to about 6 days, about 4 days to about 5 days, about 5 days to about 7 days, about 5 days to about 6 days, or about 6 days to about 7 days) after fertilization. In some embodiments, a pre-implantation embryo is an embryo that was fertilized in vivo (e.g., by intrauterine insemination). In some embodiments, a pre-implantation embryo is an embryo that was fertilized in vitro. In some embodiments, a pre-implantation embryo is a blastocyst (i.e. an embryo of 5-7 days post fertilization). In some embodiments, the pre-implantation embryo was genetically profiled by pre-implantation genetic screening and/or diagnosis prior to transfer to the uterus of a subject identified as having a receptive endometrial status. In some embodiments, a pre-implantation embryo is an embryo that can be frozen between the time of fertilization and the time of implantation.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic showing the endometrial phases during the menstrual cycle. The endometrium undergoes hormonal cyclic changes to prepare for embryo implantation. In a natural cycle the WOI usually occurs seven days following the luteinizing hormone (LH) surge (LH+7). However, in some women the WOI is displaced, occurring earlier on days 5-6 or later on days 8-9 following the LH surge.

FIG. 2 is a flow chart describing the steps involved in endometrial receptivity (ER) testing based on gene expression analysis. Following endometrial biopsy, RNA is extracted and transcribed. The resulting DNA product is used for gene expression analysis. Transcript quantities are then translated into an ER status which serves as a basis for a personalized/precision embryo transfer (PET).

FIG. 3 is a schematic showing an example of timing of an endometrial biopsy from the uterine fundus in a natural, modified natural or hormone replacement therapy (HRT) cycle.

FIG. 4 is a schematic showing the generation of an exemplary predictor consisting of four different categories: non-receptive, pre-receptive, receptive and post-receptive. In the exemplary schematic biopsies are obtained in a natural cycle. Biopsies obtained during the proliferative phase are classified as being non-receptive. Biopsies obtained on day-7 following the LH surge are classified as being receptive, whereas those obtained prior to and after day-7 are grouped into pre-receptive and post-receptive categories, respectively.

FIG. 5 is an exemplary decision tree showing treatment plan following endometrial receptivity (ER) testing.

FIG. 6 is a schematic showing an exemplary endometrial receptivity panel A array design. A total of 48 samples (one sample per sub-array) can be processed simultaneously on each array. Expression of a total of 56 genes are assessed per sample, of which eight serve as potential housekeeping/control genes (purple) and 48 as informative genes (orange).

### DETAILED DESCRIPTION

The present disclosure is based, in part, on the discovery that it is possible to determine the endometrial receptivity status of a subject using quantitative real-time polymerase chain reaction (real-time qPCR), sequencing, array hybridization, or mRNA quantification for facilitating embryo transfer by analysis of 48 genes involved in facilitating embryo implantation, endometrial proliferation and maternal immune response, or other cellular or physiological functions.

Despite continuous improvements in ART technologies, it remains the case that most IVF cycles do not result in a life birth (European IVF-Monitoring Consortium, Hum Reprod 2016; 31(8):1638-1652). In fact, the majority of embryos transferred to the uterus fail to implant or miscarry during or shortly after implantation. Successful initiation and completion of the implantation process is the result of a molecular dialogue between a viable implantation-competent embryo and a synchronised receptive endometrium, the inner lining of the uterus. Presence of a non-viable embryo or lack of endometrial synchronisation leads to implantation failure (Achache and Revel, Hum Reprod Update 2006; 12:731-746). The period during the menstrual cycle when the endometrium is receptive for an embryo to implant is known as the window of implantation (WOI) which can last up to two days (Strowitzki et al., Hum Reprod Update 2006; 12(5): 617-630). In a natural cycle or modified natural cycle the WOI commonly occurs seven days following the luteinizing hormone (LH) surge or the administration of human chorionic gonadotrophin (hCG), respectively. In a hormone replacement therapy (HRT) cycle the WOI usually occurs five days following administration of progesterone (P4) (Kalem et al., J Turk Ger Gynecol Assoc 2016; 17(3): 168-172).

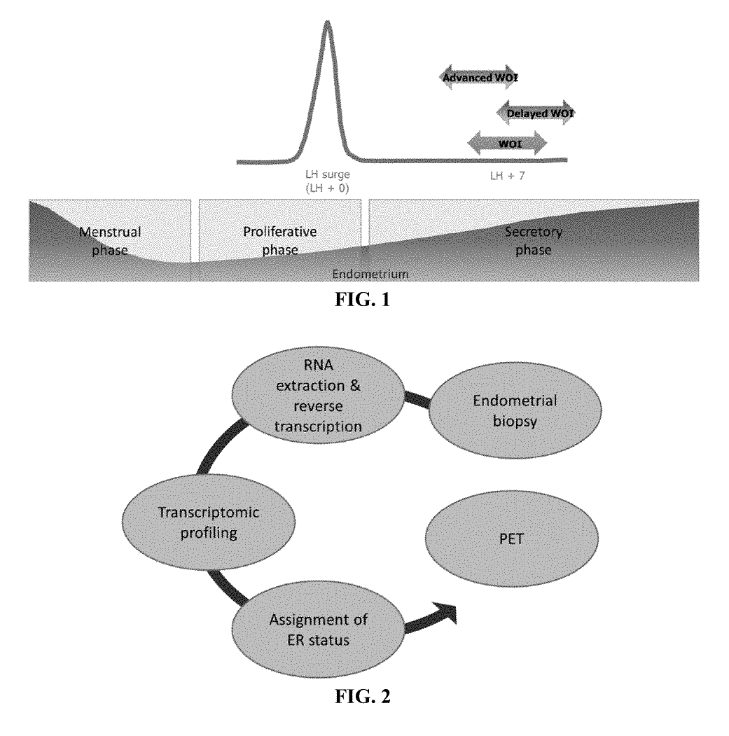

It is estimated that up to 30% of patients with recurrent IVF failure (RIF; defined as two or more failed IVF cycles and/or failed embryo transfers) have a displaced WOI which occurs either one or two days earlier (advanced WOI) or later (delayed WOI) as compared to fertile women (Ruiz-Alonso et al., Fertil Steril 2013; 100(3): 818-824). For example, in a natural cycle the WOI usually occurs seven days following the LH surge (LH+7). However, in some women the WOI is displaced, occurring earlier on days 5-6 or later on days 8-9 following the LH surge (FIG. 1). To avoid embryo transfer at a time when the endometrium is not receptive in RIF patients who generate viable implantation-competent embryos, a patient-specific WOI can be identified before initiating IVF treatment, followed by a personalised/precision embryo transfer (PET) performed during the previously identified patient-specific WOI (Ruiz-Alonso et al., Fertil Steril 2013; 100(3): 818-824).

The evaluation of endometrial receptivity (ER) has been performed for several decades via histological inspection of morphological features present in biopsied endometrial samples (Noyes et al., Fertil Steril 1950; 1:3-25). However, due to frequent inter- and intra-observer as well as inter-cycle variations, ER testing based on morphological criteria is outdated (Coutifaris et al., Fertil steril 2004; 82:1264-1272; Murray et al., Fertil Steril 2004; 98:302-307). Instead, ER testing based on gene expression analysis as described herein provides objective molecular diagnosis with highly accurate and reproducible results and an effective strategy to improve clinical outcomes in RIF patients.

The present disclosure relates to methods useful for the characterization of (e.g., clinical evaluation, diagnosis, classification, prediction, or profiling) of endometrial receptivity based on the gene expression profile of a panel of genes (e.g., panel A). The panel of genes described herein are particularly useful for characterizing (e.g., assessing or predicting) a subject for having a receptive status for embryonic implantation. Thus, in some aspects, the disclosure provides methods that include determining the gene expression profile of a selected panel of genes in a biological sample obtained from a subject, wherein a panel comprises a plurality of genes associated with endometrial receptivity. The number of genes in the plurality of genes (e.g., at least two) of panel A may be two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven, forty-eight, forty-nine, fifty, fifty-five, sixty, sixty-five, seventy, seventy-five, eighty, eighty-five, ninety, ninety-five, one hundred, one hundred and ten, one hundred and twenty, one hundred and thirty, one hundred and forty, one hundred and fifty, one hundred and sixty, one hundred and seventy, one hundred and eighty, one hundred and ninety, two hundred, or more.

Moreover, the methods described herein are useful for diagnosing whether a subject has a receptive endometrial status, a non-receptive endometrial status, a pre-receptive endometrial status, a post-receptive endometrial status, or any sub-status of endometrial receptivity. As used herein, diagnosing includes both diagnosing and aiding in diagnosing. Thus, other diagnostic criteria may be evaluated in conjunction with the results of the methods described herein in order to make a diagnosis.

The disclosure further provides for the communication of the results of the methods described herein to, e.g., technicians, physicians, nurse practitioner or patients. In some embodiments of any of the methods described herein, the method further includes communicating the endometrial status (i.e. as having a receptive endometrial status, as having a non-receptive endometrial status, as having a pre-receptive endometrial status, as having a post-receptive endometrial status) as a report. Any of the methods described herein can include a step of generating or outputting a report providing the results of any of the methods described herein. This report can be provided in the form of a tangible medium (e.g., a report printed on a paper or other tangible medium), in the form of an electronic medium (e.g., an electronic display on a computer monitor), or communicated by phone. In some embodiments, computers are used to communicate results of the methods described herein or predictions, or both, to interested parties, e.g., physicians and their patients.

The methods described herein can be used alone or in combination with other clinical methods for endometrial receptivity stratification known in the art to provide a diagnosis, a prognosis, or a prediction of endometrial receptivity. For example, clinical parameters that are known in the art for predicting endometrial receptivity may be incorporated into the analysis of one of ordinary skill in the art to arrive at an endometrial receptivity assessment with any of the methods described herein.

### Methods of Predicting

Also provided herein are methods of predicting endometrial receptivity for embryonic implantation in a human subject that include: (a) providing a first biological sample obtained from a human subject at a first time point within a menstrual cycle; (b) determining the gene expression profile of a panel of genes in the first biological sample, wherein the panel of genes comprises two or more genes selected from the group consisting of: Annexin A4 (ANXA4), Apolipoprotein D (APOD), Apolipoprotein L2 (APOL2), Aquaporin-3 (AQP3), Amphiregulin (AREG), Arginase-2 (ARG2), ATP synthase F1 subunit beta (ATP5F1B), Calpain-6 (CAPN6), Corepressor interacting with RBPJ (CIR1), CKLF-like MARVEL transmembrane domain-containing protein 5 (CMTM5), Catenin alpha 2 (CTNNA2), C-X-C motif chemokine ligand 1 (CXCL1), C-X-C motif chemokine ligand 6 (CXCL6), Estrogen receptor 1 (ESR1), Forkhead box P3 (FOXP3), Growth arrest and DNA damage-inducible alpha (GADD45A), Gap junction protein alpha 4 (GJA4), Glutathione peroxidase 3 (GPX3), Hemoglobin subunit alpha (HBA1), Hemoglobin subunit gamma-1 (HBG1), Hydroxymethylbilane synthase (HMBS), Homeobox B7 (HOXB7), Insulin-like growth factor-binding protein 1 (IGFBP1), Interleukin-1 Receptor type 1 (IL1R1), Interleukin-4 (IL4), Interleukin-5 (IL5), Integrin subunit alpha-9 (ITGA9), Integrin beta-1 (ITGB1), Monoamine oxidase A (MAOA), Mitogen-activated protein kinase 1 (MAPK1), Matrix Metallopeptidase 9 (MMP9), Metallothionein-1E (MT1E), Metallothionein-1F (MT1F), Metallothionein-1H (MT1H), Metallothionein-1L (MT1L), Metallothionein-1X (MT1X), NF-kappa-B inhibitor alpha (NFKBIA), Nicotinamide N-methyltransferase (NMMT), Progesterone Receptor (PGR), progesterone receptor membrane component 1 (PGRMC1), phospholipase A2 group IVA (PLA2G4A), Prostaglandin-endoperoxidase synthase 1 (PTGS1), Ras homolog family member A (RHOA), ribosomal protein L13a (RPL13A), Secretoglobin family 2A member 2 (SCGB2A2), Succinate dehydrogenase complex flavoprotein subunit A (SDHA), Serpin family A member 1 (SERPINA1), and Trefoil factor 3 (TFF3) using real-time qPCR analysis, sequencing, array hybridization, or any mRNA quantification method known in the art; and (c) identifying the human subject as having: (i) a receptive endometrial status, wherein the determined gene expression profile corresponds to a gene expression profile of the panel of genes of a receptive endometrial receptivity reference group (ii) a non-receptive endometrial status, wherein the determined gene expression profile corresponds to a gene expression profile of the panel of genes of a non-receptive endometrial reference group, (iii) a pre-receptive endometrial status, wherein the determined gene expression profile corresponds to a gene expression profile of the panel of genes of a pre-receptive endometrial reference group, or (iv) a post-receptive endometrial status, wherein the determined gene expression profile corresponds to a gene expression profile of the panel of genes of a post-receptive endometrial receptivity reference group.

In some aspects, the methods can include transferring pre-implantation embryo into the identified human subject. In other aspects, the methods can include obtaining a second biological sample from the human subject at a second time point and repeating steps (b) and (c) on the second biological sample.

### Methods of Determining

As used herein, an endometrial gene expression profile comprising two or more of the selected 48 genes (i.e. panel A) can be determined using any quantitative real-time PCR machine (e.g., QuantStudio™ 12K Flex Real-Time PCR System (Thermo Fisher Scientific)). In some aspects, determining an endometrial gene expression profile of a biological sample (e.g., an endometrial biopsy sample) can include: extracting RNA from the biological sample, performing reverse transcription to generate cDNA, contacting the generated cDNA with pairs of primers targeting the genes of panel A and the control genes, collecting gene expression data using real-time qPCR analysis software, applying classification models/algorithms to determine the endometrial receptivity status of the biological sample as compared to the gene expression profile of panel A of a reference group (e.g., the receptive endometrial reference group, the non-receptive endometrial reference group, the pre-receptive endometrial reference group, the post-receptive endometrial reference group).

In some embodiments, the expression of a panel of genes can be also be assessed by determining the expression level of proteins encoded by each gene in the panel (Table 1).

TABLE 1
Non-Limiting Examples of Panel
Gene mRNA and Protein Sequences

| | mRNA NCBI | Protein NCBI |
|---|---|---|
| Gene Symbol | Accession No. | Accession No. |
| ANXA4 | NM_001320698.1 | NP_001144.1 |
| APOD | NM_001647.3 | NP_001638.1 |
| APOL2 | NM_030882.3 | NP_112092.2 |
| AQP3 | NM_004925.4 | NP_004916.1 |
| AREG | NM_001657.3 | NP_001648.1 |
| ARG2 | NM_001172.3 | NP_001163.1 |
| ATP5F1B | NM_001686.3 | NP_001677.2 |
| CAPN6 | NM_014289.3 | NP_055104.2 |
| CIR1 | NM_004882.3 | NP_004873.3 |
| CMTM5 | NM_138460.2 | NP_612469.1 |
| CTNNA2 | NM_001282597.2 | NP_001269526.1 |
| CXCL1 | NM_001511.3 | NP_001502.1 |
| CXCL6 | NM_002993.3 | NP_002984.1 |
| ESR1 | NM_000125.3 | NP_000116.2 |
| FOXP3 | NM_014009.3 | NP_054728.2 |
| GADD45A | NM_001924.3 | NP_001915.1 |
| GJA4 | NM_002060.2 | NP_002051.2 |
| GPX3 | NM_002084.4 | NP_002075.2 |
| HBA1 | NM_000558.4 | NP_000549.1 |
| HBG1 | NM_000559.2 | NP_000550.2 |
| HMBS | NM_000190.3 | NP_000181.2 |
| HOXB7 | NM_004502.3 | NP_004493.3 |
| IGFBP1 | NM_000596.3 | NP_000587.1 |
| IL1R1 | NM_001288706.1 | NP_001275635.1 |
| IL4 | NM_000589.3 | NP_000580.1 |
| IL5 | NM_000879.2 | NP_000870.1 |
| ITGA9 | NM_002207.2 | NP_002198.2 |
| ITGB1 | NM_002211.3 | NP_002202.2 |
| MAOA | NM_000240.3 | NP_000231.1 |
| MAPK1 | NM_002745.4 | NP_002736.3 |
| MMP9 | NM_004994.2 | NP_004985.2 |
| MT1E | NM_175617.3 | NP_783316.2 |
| MT1F | NM_005949.3 | NP_005940.1 |
| MT1H | NM_005951.2 | NP_005942.1 |
| MT1L | NR_001447.2 | |
| MT1X | NM_005952.3 | NP_005943.1 |
| NFKBIA | NM_020529.2 | NP_065390.1 |
| NNMT | NM_006169.2 | NP_006160.1 |
| PGR | NM_000926.4 | NP_000917.3 |
| PGRMC1 | NM_006667.4 | NP_006658.1 |
| PLA2G4A | NM_024420.2 | NP_077734.1 |
| PTGS1 | NM_000962.3 | NP_000953.2 |
| RHOA | NM_001664.3 | NP_001655.1 |
| RPL13A | NM_012423.3 | NP_036555.1 |
| SCGB2A2 | NM_002411.3 | NP_002402.1 |
| SDHA | NM_004168.3 | NP_004159.2 |
| SERPINA1 | NM_000295.4 | NP_000286.3 |
| TFF3 | NM_003226.3 | NP_003217.3 |

Each reverse transcription PCR reaction occurs in a reaction volume that includes all of the components required to carry out a reaction, e.g., primers, buffer, DNA polymerase, reverse transcriptase, sample. The determining step of each gene within the panel of genes is performed in a reaction volume of 0.005 μL to 100 μL (e.g., 0.005 μL to 100 μL, 0.005 μL to 90 μL, 0.005 μL to 80 μL, 0.005 μL to 70 μL, 0.005 μL to 60 μL, 0.005 μL to 50 μL, 0.005 μL to 40 μL, 0.005 μL to 30 μL, 0.005 μL to 20 μL, 0.005 μL to 10 μL, 0.01 μL to 100 μL, 0.01 μL to 90 μL, 0.01 μL to 80 μL, 0.01 μL to 70 μL, 0.01 μL to 60 μL, 0.01 μL to 50 μL, 0.01 μL to 40 μL, 0.01 μL to 30 μL, 0.01 μL to 20 μL, 0.01 μL to 10 μL, 0.02 μL to 100 μL, 0.02 μL to 90 μL, 0.02 μL to 80 μL, 0.02 μL to 70 μL, 0.02 μL to 60 μL, 0.02 μL to 50 μL, 0.02 μL to 40 μL, 0.02 μL to 30 μL, 0.02 μL to 20 μL, 0.02 μL to 10 μL, 0.02 μL to 0.05 μL, 0.02 μL to 0.04 μL, 0.05 μL to 100 μL, 0.05 μL to 90 μL, 0.05 μL to 80 μL, 0.05 μL to 70 μL, 0.05 μL to 60 μL, 0.05 μL to 50 μL, 0.05 μL to 40 μL, 0.05 μL to 30 μL, 0.05 μL to 20 μL, 0.05 μL to 10 μL, 1 μL to 100 μL, 1 μL to 90 μL, 1 μL to 80 μL, 1 μL to 70 μL, 1 μL to 60 μL, 1 μL to 50 μL, 1 μL to 40 μL, 1 μL to 30 μL, 1 μL to 20 μL, 1 μL to 10 μL, 5 μL to 100 μL, 5 μL to 90 μL, 5 μL to 80 μL, 5 μL to 70 μL, 5 μL to 60 μL, 5 μL to 50 μL, 5 μL to 40 μL, 5 μL to 30 μL, 5 μL to 20 μL, 5 μL to 10 μL, 10 μL to 100 μL, 10 μL to 90 μL, 10 μL to 80 μL, 10 μL to 70 μL, 10 μL to 60 μL, 10 μL to 50 μL, 10 μL to 40 μL, 10 μL to 30 μL, 10 μL to 20 μL, 20 μL to 50 μL, 20 μL to 40 μL, 15 μL to 100 μL, 15 μL to 90 μL, 15 μL to 80 μL, 15 μL to 70 μL, 15 μL to 60 μL, 15 μL to 50 μL, 15 μL to 40 μL, 15 μL to 30 μL, 15 μL to 20 μL, 20 μL to 100 μL, 20 μL to 90 μL, 20 μL to 80 μL, 20 μL to 70 μL, 20 μL to 60 μL, 20 μL to 50 μL, 20 μL to 40 μL, 20 μL to 30 μL, 50 μL to 100 μL, 50 μL to 90 μL, 50 μL to 80 μL, 50 μL to 70 μL, 50 μL to 60 μL, 25 μL to 100 μL, 30 μL to 100 μL, 40 μL to 100 μL, 50 μL to 100 μL, 60 μL to 100 μL, 70 μL to 100 μL, 80 μL to 100 μL, 90 μL to 100 μL).

Methods of digesting a tissue sample (e.g., an endometrial biopsy sample) and extracting RNA from a tissue sample are well-known in the art and are described herein.

As used herein, the term “principal component analysis” or “principal component algorithm” refers to a statistical method that uses an orthogonal transformation to convert a set of observations of possibly correlated variables into a set of values of linearly uncorrelated variables called principal components. It finds the principal components of the dataset and transforms the data into a new, lower-dimensional subspace. The principle component, which can be represented by an eigenvector, mathematically corresponds to a direction in the original n-dimensional space, so that the first principal component accounts for as much of the variance in the data as possible, and each succeeding component accounts for as much of the remaining variance as possible.

Principal component analysis (PCA) is a statistical method that uses an orthogonal transformation to convert a set of observations of possibly correlated variables into a set of values of linearly uncorrelated variables called principal components. It finds the principal components of the dataset and transforms the data into a new, lower-dimensional subspace. The transformation is defined in such a way that the first principal component has the largest possible variance (that is, accounts for as much of the variability in the data as possible), and each succeeding component in turn has the highest variance possible under the constraint that it is orthogonal to the preceding components. The resulting vectors are an uncorrelated orthogonal basis set. PCA is sensitive to the relative scaling of the original variables.

Mathematically, the principal components are the eigenvectors of the covariance or correlation matrix of the original dataset. As the covariance matrix or correlation matrix is symmetric, the eigenvectors are orthogonal. The principal components (eigenvectors) correspond to the direction (in the original n-dimensional space) with the greatest variance in the data. Each eigenvector has a corresponding eigenvalue. An eigenvalue is a scalar. The corresponding eigenvalue is a number that indicates how much variance there is in the data along that eigenvector (or principal component). A large eigenvalue means that that principal component explains a large amount of the variance in the data. Similarly, a principal component with a very small eigenvalue explains a small amount variance in the data.

Detailed descriptions regarding how to perform PCA are described in numerous references, e.g., Smith, Lindsay I. “A tutorial on principal components analysis.” Cornell University, USA 51 (2002): 52; Shlens, Jonathon. “A tutorial on principal component analysis.” arXiv preprint arXiv:1404.1100 (2014), each of which is incorporated by reference in its entirety.

For example, to apply principle component analysis for the disclosed methods, a set of data comprising expression profile of a panel of genes is created for each sample. The set of data for a sample can be represented by a vector. The dataset can include the expression profile for all subjects in reference group of interest (e.g., a receptive endometrial reference group, a non-receptive endometrial reference group, a pre-receptive endometrial reference group, a post-receptive endometrial reference group) and/or the expression profile of the panel of the genes for tested subjects. The principal component analysis (PCA) converts the dataset into a dataset with lower dimensions. The positions of the each subject (including subjects in the reference group and the tested subject) are determined in this lower dimensional space. In this lower dimension space, if the tested subject is closer to, or is clustered with a particular reference group, then it can be determined that this tested subject corresponds to this particular reference group.

The methods to determine whether a test subject is closer to, or is clustered with, a particular reference group are known in the art, and can be determined by algorithms known in the art, e.g., hierarchical clustering algorithm, k-means clustering algorithm, a statistical distribution model, etc. Various computer algorithms for data analysis and classification are known in the art to compare gene expression profiles. See, e.g., Diaz-Gimeno et al., Fertil Steril 2011 95(1): 50-60; Diaz-Gimeno et al., Fertil Steril 2013; 99: 508-517.

### Methods of Identifying

Also provided herein are methods of identifying a human subject (e.g., any of the human subjects described herein) as having:
(i) a receptive endometrial status, when the determined gene expression profile corresponds to a gene expression profile of the panel of genes of a receptive endometrial receptivity reference group,
(ii) a non-receptive endometrial status, when the determined gene expression profile corresponds to a gene expression profile of the panel of genes of a non-receptive endometrial receptivity reference group,
(iii) a pre-receptive endometrial status, when the determined gene expression profile corresponds to a gene expression profile of the panel of genes of a pre-receptive endometrial receptivity reference group, or
(iv) a post-receptive endometrial status, when the determined gene expression profile corresponds to a gene expression profile of the panel of genes of a post-receptive endometrial receptivity reference group.

In some embodiments of any of the methods described herein, the method further includes after identifying the human subject as having a receptive endometrial status, (d) transferring a pre-implantation embryo into the identified human subject.

In some embodiments of any of the methods described herein, the method further includes after identifying the human subject as having a non-receptive endometrial status, a pre-receptive endometrial status, or a post-receptive endometrial status, (d) obtaining a second biological sample from the human subject at a second time point and repeating steps (b) and (c) on the second biological sample. In some embodiments, the method further includes after identifying the human subject as having a receptive endometrial status, (e) transferring a pre-implantation embryo into the identified human subject. In some embodiments, the second biological sample is an endometrial biopsy obtained from the uterine fundus.

In some embodiments of any of the methods described herein, the human subject is identified as having a post-receptive endometrial status, and the second biological sample is obtained from the human subject in another menstrual cycle, one or two days before the first biological sample was obtained from the human subject in the previous menstrual cycle.

In some embodiments of any of the methods described herein, the human subject is identified as having a pre-receptive endometrial status, and the second biological sample is obtained from the human subject in another menstrual cycle, one or two days after the first biological sample was obtained from the human subject in the previous menstrual cycle.

In some embodiments of any of the methods described herein wherein the human subject is identified as having a non-receptive endometrial status, the method further includes instructing a healthcare professional to select a treatment plan for the identified human subject.

In some embodiments of any of the methods described herein wherein the human subject is identified as having a non-receptive endometrial status, the method further includes selecting a treatment plan for the identified human subject. In some embodiments, the treatment plan includes a hormone replacement therapy cycle.

### Genes Useful for Predicting Endometrial Receptivity

The methods provided herein include the determination of the gene expression profile of a panel of genes, wherein the panel comprises two more genes selected from the group consisting of: ANXA4, APOD, APOL2, AQP3, AREG, ARG2, ATP5F1B, CAPN6, CIR1, CMTM5, CTNNA2, CXCL1, CXCL6, ESR1, FOXP3, GADD45A, GJA4, GPX3, HBA1, HBG1, HMBS, HOXB7, IGFBP1, IL1R1, IL4, IL5, ITGA9, ITGB1, MAOA, MAPK1, MMP9, MT1E, MT1F, MT1H, MT1L, MT1X, NFKBIA, NMMT, PGR, PGRMC1, PLA2G4A, PTGS1, RHOA, RPL13A, SCGB2A2, SDHA, SERPINA1 and TFF3, in at least one sample from a human subject.

A panel of any combination of genes described herein can be determined in a biological sample (e.g., any of the biological samples described herein) in any of the methods described herein. Examples of combinations of genes that can be determined (e.g., the expression profile determined) in a biological sample (e.g., any of the biological samples described herein) in any of the methods described herein are shown in Table 2.

TABLE 2
Non-limiting Examples of Gene Panels

APOD, APOL2
APOD, APOL2, AQP3
APOD, APOL2, AQP3, AREG
APOD, APOL2, AQP3, AREG, ARG2
APOD, APOL2, AQP3, AREG, ARG2, ATP5F1B
APOD, APOL2, AQP3, AREG, ARG2, ATP5F1B, CAPN6
APOD, APOL2, AQP3, AREG, ARG2, ATP5F1B, CAPN6, CIR1
APOD, APOL2, AQP3, AREG, ARG2, ATP5F1B, CAPN6, CIR1, CMTM5
APOD, APOL2, AQP3, AREG, ARG2, ATP5F1B, CAPN6, CIR1, CMTM5, CTNNA2
APOD, APOL2, AQP3, AREG, ARG2, ATP5F1B, CAPN6, CIR1, CMTM5, CTNNA2, CXCL1
APOD, APOL2, AQP3, AREG, ARG2, ATP5F1B, CAPN6, CIR1, CMTM5, CTNNA2, CXCL1,
CXCL6
APOD, APOL2, AQP3, AREG, ARG2, ATP5F1B, CAPN6, CIR1, CMTM5, CTNNA2, CXCL1,
CXCL6, ESR1
APOD, APOL2, AQP3, AREG, ARG2, ATP5F1B, CAPN6, CIR1, CMTM5, CTNNA2, CXCL1,
CXCL6, ESR1, FOXP3
APOD, APOL2, AQP3, AREG, ARG2, ATP5F1B, CAPN6, CIR1, CMTM5, CTNNA2, CXCL1,
CXCL6, ESR1, FOXP3, GADD45A
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
APOD, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD
ANXA4, APOD, APOL2
ANXA4, APOD, APOL2, AQP3
ANXA4, APOD, APOL2, AQP3, AREG
ANXA4, APOD, APOL2, AQP3, AREG, ARG2
ANXA4, APOD, APOL2, AQP3, AREG, ARG2, ATP5F1B
ANXA4, APOD, APOL2, AQP3, AREG, ARG2, ATP5F1B, CAPN6
ANXA4, APOD, APOL2, AQP3, AREG, ARG2, ATP5F1B, CAPN6, CIR1
ANXA4, APOD, APOL2, AQP3, AREG, ARG2, ATP5F1B, CAPN6, CIR1, CMTM5
ANXA4, APOD, APOL2, AQP3, AREG, ARG2, ATP5F1B, CAPN6, CIR1, CMTM5, CTNNA2
ANXA4, APOD, APOL2, AQP3, AREG, ARG2, ATP5F1B, CAPN6, CIR1, CMTM5, CTNNA2,
CXCL1
ANXA4, APOD, APOL2, AQP3, AREG, ARG2, ATP5F1B, CAPN6, CIR1, CMTM5, CTNNA2,
CXCL1, CXCL6
ANXA4, APOD, APOL2, AQP3, AREG, ARG2, ATP5F1B, CAPN6, CIR1, CMTM5, CTNNA2,
CXCL1, CXCL6, ESR1
ANXA4, APOD, APOL2, AQP3, AREG, ARG2, ATP5F1B, CAPN6, CIR1, CMTM5, CTNNA2,
CXCL1, CXCL6, ESR1, FOXP3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA, SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA, SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1, RHOA, RPL13A, SCGB2A2,
SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1, RHOA, RPL13A, SCGB2A2,
SDHA, SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGRMC1, PLA2G4A, PTGS1, RHOA, RPL13A, SCGB2A2,
SDHA, SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA, SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1H,
MT1L, MT1X, NFKBIA, NMMT, PGR, PGRMC1, PLA2G4A, PTGS1, RHOA, RPL13A, SCGB2A2,
SDHA, SERPINA1,
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1F, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1E, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MMP9, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MAPK1, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAOA, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, ITGB1, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGA9, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, IL5, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL4, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL1R1, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7,
IGFBP1, IL4, IL5, ITGA9, ITGB1, MAOA, MAPK1, MMP9, MT1E, MT1F, MT1H, MT1L,
MT1X, NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, HOXB7, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HMBS, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HBG1, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBA1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, GPX3, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GJA4, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GADD45A, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1, IL4
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, FOXP3, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, ESR1, GADD45A
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, ESR1, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, CXCL6, FOXP3
ANXA4, APOD, APOL2, AQP3, AREG, ARG2, ATP5F1B, CAPN6, CIR1, CMTM5, CTNNA2,
CXCL1, CXCL6, FOXP3, GADD45A
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, CXCL6, FOXP3, GADD45A, GJA4, GPX3, HBA1, HBG1, HMBS, HOXB7, IGFBP1,
IL1R1, IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL1, ESR1
ANXA4, APOD, APOL2, AQP3, AREG, ARG2, ATP5F1B, CAPN6, CIR1, CMTM5, CTNNA2,
CXCL1, ESR1, FOXP3
ANXA4, APOD, APOL2, AQP3, AREG, ARG2, ATP5F1B, CAPN6, CIR1, CMTM5, CTNNA2,
CXCL1, ESR1, FOXP3, GADD45A
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL1, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CTNNA2,
CXCL6
ANXA4, APOD, APOL2, AQP3, AREG, ARG2, ATP5F1B, CAPN6, CIR1, CMTM5, CTNNA2,
CXCL6, ESR1
ANXA4, APOD, APOL2, AQP3, AREG, ARG2, ATP5F1B, CAPN6, CIR1, CMTM5, CTNNA2,
CXCL6, ESR1, FOXP3
ANXA4, APOD, APOL2, AQP3, AREG, ARG2, ATP5F1B, CAPN6, CIR1, CMTM5, CTNNA2,
CXCL6, ESR1, FOXP3, GADD45A
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CTNNA2,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CMTM5, CXCL1
ANXA4, APOD, APOL2, AQP3, AREG, ARG2, ATP5F1B, CAPN6, CIR1, CMTM5, CXCL1,
CXCL6
ANXA4, APOD, APOL2, AQP3, AREG, ARG2, ATP5F1B, CAPN6, CIR1, CMTM5, CXCL1,
CXCL6, ESR1
ANXA4, APOD, APOL2, AQP3, AREG, ARG2, ATP5F1B, CAPN6, CIR1, CMTM5, CXCL1,
CXCL6, ESR1, FOXP3
ANXA4, APOD, APOL2, AQP3, AREG, ARG2, ATP5F1B, CAPN6, CIR1, CMTM5, CXCL1,
CXCL6, ESR1, FOXP3, GADD45A
ANXA4, APOD, APOL2, AQP3, AREG, ARG2, ATP5F1B, CAPN6, CIR1, CMTM5, CXCL1,
CXCL6, ESR1, FOXP3, GADD45A, GJA4
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CMTM5, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CIR1, CTNNA2
ANXA4, APOD, APOL2, AQP3, AREG, ARG2, ATP5F1B, CAPN6, CIR1, CTNNA2, CXCL1
ANXA4, APOD, APOL2, AQP3, AREG, ARG2, ATP5F1B, CAPN6, CIR1, CTNNA2, CXCL1,
CXCL6
ANXA4, APOD, APOL2, AQP3, AREG, ARG2, ATP5F1B, CAPN6, CIR1, CTNNA2, CXCL1,
CXCL6, ESR1
ANXA4, APOD, APOL2, AQP3, AREG, ARG2, ATP5F1B, CAPN6, CIR1, CTNNA2, CXCL1,
CXCL6, ESR1, FOXP3
ANXA4, APOD, APOL2, AQP3, AREG, ARG2, ATP5F1B, CAPN6, CIR1, CTNNA2, CXCL1,
CXCL6, ESR1, FOXP3, GADD45A
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CIR1, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CAPN6, CMTM5
ANXA4, APOD, APOL2, AQP3, AREG, ARG2, ATP5F1B, CAPN6, CMTM5, CTNNA2
ANXA4, APOD, APOL2, AQP3, AREG, ARG2, ATP5F1B, CAPN6, CMTM5, CTNNA2, CXCL1
ANXA4, APOD, APOL2, AQP3, AREG, ARG2, ATP5F1B, CAPN6, CMTM5, CTNNA2, CXCL1,
CXCL6
ANXA4, APOD, APOL2, AQP3, AREG, ARG2, ATP5F1B, CAPN6, CMTM5, CTNNA2, CXCL1,
CXCL6, ESR1
ANXA4, APOD, APOL2, AQP3, AREG, ARG2, ATP5F1B, CAPN6, CMTM5, CTNNA2, CXCL1,
CXCL6, ESR1, FOXP3
ANXA4, APOD, APOL2, AQP3, AREG, ARG2, ATP5F1B, CAPN6, CMTM5, CTNNA2, CXCL1,
CXCL6, ESR1, FOXP3, GADD45A
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CAPN6, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, ATP5F1B, CIR1
ANXA4, APOD, APOL2, AQP3, AREG, ARG2, ATP5F1B, CIR1, CMTM5
ANXA4, APOD, APOL2, AQP3, AREG, ARG2, ATP5F1B, CIR1, CMTM5, CTNNA2
ANXA4, APOD, APOL2, AQP3, AREG, ARG2, ATP5F1B, CIR1, CMTM5, CTNNA2, CXCL1
ANXA4, APOD, APOL2, AQP3, AREG, ARG2, ATP5F1B, CIR1, CMTM5, CTNNA2, CXCL1,
CXCL6
ANXA4, APOD, APOL2, AQP3, AREG, ARG2, ATP5F1B, CIR1, CMTM5, CTNNA2, CXCL1,
CXCL6, ESR1
ANXA4, APOD, APOL2, AQP3, AREG, ARG2, ATP5F1B, CIR1, CMTM5, CTNNA2, CXCL1,
CXCL6, ESR1, FOXP3
ANXA4, APOD, APOL2, AQP3, AREG, ARG2, ATP5F1B, CIR1, CMTM5, CTNNA2, CXCL1,
CXCL6, ESR1, FOXP3, GADD45A
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, ATP5F1B, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ARG2, CAPN6
ANXA4, APOD, APOL2, AQP3, AREG, ARG2, CAPN6, CIR1
ANXA4, APOD, APOL2, AQP3, AREG, ARG2, CAPN6, CIR1, CMTM5
ANXA4, APOD, APOL2, AQP3, AREG, ARG2, CAPN6, CIR1, CMTM5, CTNNA2
ANXA4, APOD, APOL2, AQP3, AREG, ARG2, CAPN6, CIR1, CMTM5, CTNNA2, CXCL1
ANXA4, APOD, APOL2, AQP3, AREG, ARG2, CAPN6, CIR1, CMTM5, CTNNA2, CXCL1, CXCL6
ANXA4, APOD, APOL2, AQP3, AREG, ARG2, CAPN6, CIR1, CMTM5, CTNNA2, CXCL1,
CXCL6, ESR1
ANXA4, APOD, APOL2, AQP3, AREG, ARG2, CAPN6, CIR1, CMTM5, CTNNA2, CXCL1,
CXCL6, ESR1, FOXP3
ANXA4, APOD, APOL2, AQP3, AREG, ARG2, CAPN6, CIR1, CMTM5, CTNNA2, CXCL1,
CXCL6, ESR1, FOXP3, GADD45A
ANXA4, APOD, APOL2, AQP3, AREG, ARG2, CAPN6, CIR1, CMTM5, CTNNA2, CXCL1,
CXCL6, ESR1, FOXP3, GADD45A, GJA4
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ARG2, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, AREG, ATP5F1B
ANXA4, APOD, APOL2, AQP3, AREG, ATP5F1B, CAPN6
ANXA4, APOD, APOL2, AQP3, AREG, ATP5F1B, CAPN6, CIR1
ANXA4, APOD, APOL2, AQP3, AREG, ATP5F1B, CAPN6, CIR1, CMTM5
ANXA4, APOD, APOL2, AQP3, AREG, ATP5F1B, CAPN6, CIR1, CMTM5, CTNNA2
ANXA4, APOD, APOL2, AQP3, AREG, ATP5F1B, CAPN6, CIR1, CMTM5, CTNNA2, CXCL1
ANXA4, APOD, APOL2, AQP3, AREG, ATP5F1B, CAPN6, CIR1, CMTM5, CTNNA2, CXCL1,
CXCL6
ANXA4, APOD, APOL2, AQP3, AREG, ATP5F1B, CAPN6, CIR1, CMTM5, CTNNA2, CXCL1,
CXCL6, ESR1
ANXA4, APOD, APOL2, AQP3, AREG, ATP5F1B, CAPN6, CIR1, CMTM5, CTNNA2, CXCL1,
CXCL6, ESR1, FOXP3
ANXA4, APOD, APOL2, AQP3, AREG, ATP5F1B, CAPN6, CIR1, CMTM5, CTNNA2, CXCL1,
CXCL6, ESR1, FOXP3, GADD45A
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, AREG, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AQP3, ARG2
ANXA4, APOD, APOL2, AQP3, ARG2, ATP5F1B
ANXA4, APOD, APOL2, AQP3, ARG2, ATP5F1B, CAPN6
ANXA4, APOD, APOL2, AQP3, ARG2, ATP5F1B, CAPN6, CIR1
ANXA4, APOD, APOL2, AQP3, ARG2, ATP5F1B, CAPN6, CIR1, CMTM5
ANXA4, APOD, APOL2, AQP3, ARG2, ATP5F1B, CAPN6, CIR1, CMTM5, CTNNA2
ANXA4, APOD, APOL2, AQP3, ARG2, ATP5F1B, CAPN6, CIR1, CMTM5, CTNNA2, CXCL1
ANXA4, APOD, APOL2, AQP3, ARG2, ATP5F1B, CAPN6, CIR1, CMTM5, CTNNA2, CXCL1,
CXCL6
ANXA4, APOD, APOL2, AQP3, ARG2, ATP5F1B, CAPN6, CIR1, CMTM5, CTNNA2, CXCL1,
CXCL6, ESR1
ANXA4, APOD, APOL2, AQP3, ARG2, ATP5F1B, CAPN6, CIR1, CMTM5, CTNNA2, CXCL1,
CXCL6, ESR1, FOXP3
ANXA4, APOD, APOL2, AQP3, ARG2, ATP5F1B, CAPN6, CIR1, CMTM5, CTNNA2, CXCL1,
CXCL6, ESR1, FOXP3, GADD45A
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AQP3, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, APOL2, AREG
ANXA4, APOD, APOL2, AREG, ARG2
ANXA4, APOD, APOL2, AREG, ARG2, ATP5F1B
ANXA4, APOD, APOL2, AREG, ARG2, ATP5F1B, CAPN6
ANXA4, APOD, APOL2, AREG, ARG2, ATP5F1B, CAPN6, CIR1
ANXA4, APOD, APOL2, AREG, ARG2, ATP5F1B, CAPN6, CIR1, CMTM5
ANXA4, APOD, APOL2, AREG, ARG2, ATP5F1B, CAPN6, CIR1, CMTM5, CTNNA2
ANXA4, APOD, APOL2, AREG, ARG2, ATP5F1B, CAPN6, CIR1, CMTM5, CTNNA2, CXCL1
ANXA4, APOD, APOL2, AREG, ARG2, ATP5F1B, CAPN6, CIR1, CMTM5, CTNNA2, CXCL1,
CXCL6
ANXA4, APOD, APOL2, AREG, ARG2, ATP5F1B, CAPN6, CIR1, CMTM5, CTNNA2, CXCL1,
CXCL6, ESR1
ANXA4, APOD, APOL2, AREG, ARG2, ATP5F1B, CAPN6, CIR1, CMTM5, CTNNA2, CXCL1,
CXCL6, ESR1, FOXP3
ANXA4, APOD, APOL2, AREG, ARG2, ATP5F1B, CAPN6, CIR1, CMTM5, CTNNA2, CXCL1,
CXCL6, ESR1, FOXP3, GADD45A
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, APOL2, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOD, AQP3
ANXA4, APOD, AQP3, AREG
ANXA4, APOD, AQP3, AREG, ARG2
ANXA4, APOD, AQP3, AREG, ARG2, ATP5F1B
ANXA4, APOD, AQP3, AREG, ARG2, ATP5F1B, CAPN6
ANXA4, APOD, AQP3, AREG, ARG2, ATP5F1B, CAPN6, CIR1
ANXA4, APOD, AQP3, AREG, ARG2, ATP5F1B, CAPN6, CIR1, CMTM5
ANXA4, APOD, AQP3, AREG, ARG2, ATP5F1B, CAPN6, CIR1, CMTM5, CTNNA2
ANXA4, APOD, AQP3, AREG, ARG2, ATP5F1B, CAPN6, CIR1, CMTM5, CTNNA2, CXCL1
ANXA4, APOD, AQP3, AREG, ARG2, ATP5F1B, CAPN6, CIR1, CMTM5, CTNNA2, CXCL1,
CXCL6
ANXA4, APOD, AQP3, AREG, ARG2, ATP5F1B, CAPN6, CIR1, CMTM5, CTNNA2, CXCL1,
CXCL6, ESR1
ANXA4, APOD, AQP3, AREG, ARG2, ATP5F1B, CAPN6, CIR1, CMTM5, CTNNA2, CXCL1,
CXCL6, ESR1, FOXP3
ANXA4, APOD, AQP3, AREG, ARG2, ATP5F1B, CAPN6, CIR1, CMTM5, CTNNA2, CXCL1,
CXCL6, ESR1, FOXP3, GADD45A
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOD, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3
ANXA4, APOL2
ANXA4, APOL2, AQP3
ANXA4, APOL2, AQP3, AREG
ANXA4, APOL2, AQP3, AREG, ARG2
ANXA4, APOL2, AQP3, AREG, ARG2, ATP5F1B
ANXA4, APOL2, AQP3, AREG, ARG2, ATP5F1B, CAPN6
ANXA4, APOL2, AQP3, AREG, ARG2, ATP5F1B, CAPN6, CIR1
ANXA4, APOL2, AQP3, AREG, ARG2, ATP5F1B, CAPN6, CIR1, CMTM5
ANXA4, APOL2, AQP3, AREG, ARG2, ATP5F1B, CAPN6, CIR1, CMTM5, CTNNA2
ANXA4, APOL2, AQP3, AREG, ARG2, ATP5F1B, CAPN6, CIR1, CMTM5, CTNNA2, CXCL1
ANXA4, APOL2, AQP3, AREG, ARG2, ATP5F1B, CAPN6, CIR1, CMTM5, CTNNA2, CXCL1,
CXCL6
ANXA4, APOL2, AQP3, AREG, ARG2, ATP5F1B, CAPN6, CIR1, CMTM5, CTNNA2, CXCL1,
CXCL6, ESR1
ANXA4, APOL2, AQP3, AREG, ARG2, ATP5F1B, CAPN6, CIR1, CMTM5, CTNNA2, CXCL1,
CXCL6, ESR1, FOXP3
ANXA4, APOL2, AQP3, AREG, ARG2, ATP5F1B, CAPN6, CIR1, CMTM5, CTNNA2, CXCL1,
CXCL6, ESR1, FOXP3, GADD45A
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1
ANXA4, APOL2, AQP3, AREG, ARG2, ATP5F1B, CAPN6, CIR1, CMTM5, CTNNA2, CXCL1,
CXCL6, ESR1, FOXP3, GADD45A, GJA4, GPX3, HBA1, HBG1, HMBS, HOXB7, IGFBP1, IL1R1,
IL4, IL5, ITGA9, ITGB1, MAOA, MAPK1, MMP9, MT1E, MT1F, MT1H, MT1L, MT1X,
NFKBIA, NMMT, PGR, PGRMC1, PLA2G4A, PTGS1, RHOA, RPL13A, SCGB2A2, SDHA,
SERPINA1, TFF3

### Kits

Also provided herein are kits that include any of the reagents suitable for predicting endometrial receptivity for transplantation of a pre-implantation embryo. The kits include reagents suitable for determining an endometrial gene expression profile of a panel of genes (e.g., panel A). In some embodiments, the kits can include instructions for performing use of the kit in the methods described herein. In some embodiments, the reagents suitable for determining the endometrial gene expression profile of the biological sample are disposed in an array, a chip, a multi-well plate (e.g., a 96-well plate or a 384-well plate), or a tube (e.g., a 0.2 mL microcentrifuge tube). In some embodiments of any of the kits described herein, the kit includes an array, a chip, a multi-well plate (e.g., a 96-well plate or a 384-well plate), or a tube (e.g., a 0.2 mL microcentrifuge tube). In some embodiments of any of the kits described herein, the kit includes one or more reference groups (e.g., the receptive endometrial reference group, the non-receptive endometrial reference group, the pre-receptive endometrial reference group, the post-receptive endometrial reference group) for determining endometrial gene expression profile of a sample based on computer-assisted algorithms (e.g., principle component analysis, or any other classification algorithms known in the art). In some cases, the kits include software useful for comparing the endometrial gene expression profile of a sample with a reference group (e.g., a prediction model). The software may be provided in a computer readable format (e.g., a compact disc, DVD, flash drive, zip drive etc.), or the software may be available for downloading via the internet. The kits described herein are not so limited; other variations will be apparent to one of ordinary skill in the art.

### EXAMPLES

The invention is further described in the following examples, which do not limit the scope of the invention described in the claims.

### Example 1. Overview of Endometrial Receptivity Testing

As described herein, the endometrial receptivity (ER) status of a human subject is determined using real-time qPCR, and can be clinically implemented (FIG. 2). First, an endometrial biopsy is obtained from a human subject. An endometrial biopsy can be taken using any of the methods or devices known in the art, e.g., a pipette, an endometrial pipelle, an aspirating device). The endometrial tissue is digested, and the RNA is extracted. To enable gene expression analysis, reverse transcription is performed, which transcribes the single-stranded RNA molecules into complementary DNA (cDNA), which serves as a template for any subsequent sample processing step. Next, a transcriptomic profile is determined using the panel of genes described herein. Gene expression analysis using real-time qPCR is performed utilising a high-throughput real-time qPCR technology, which allows simultaneous analysis of a large number of genes. The real-time qPCR data is internally normalised and translated into relative gene quantities.

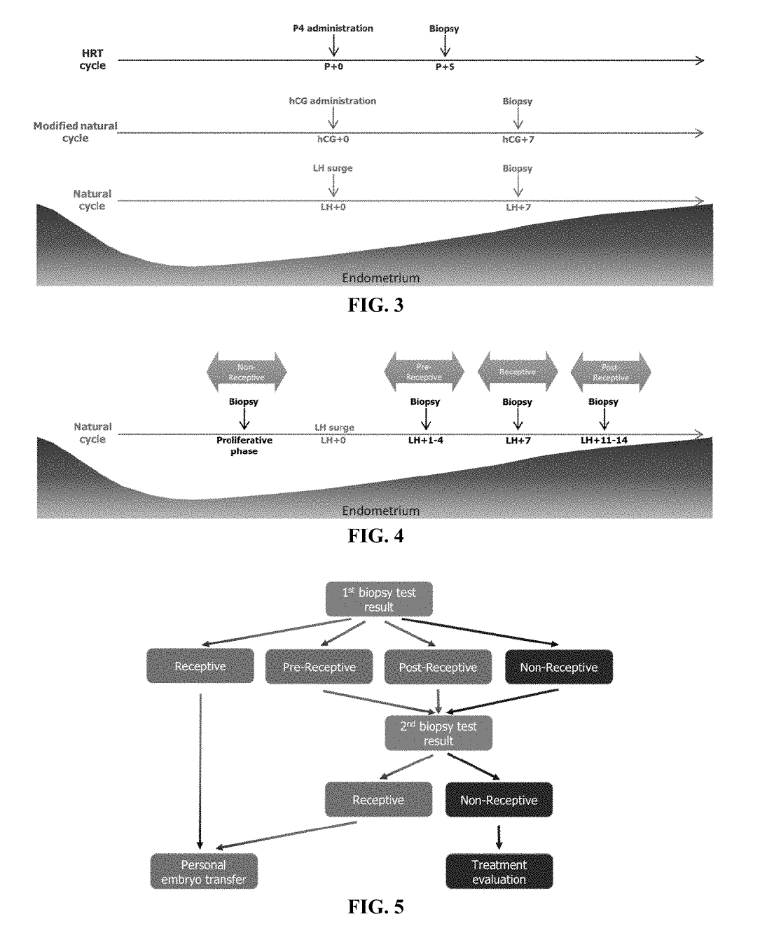

An endometrial biopsy is taken from the uterine fundus in order to estimate a window of implantation (WOI). As shown in FIG. 3, the biopsy can be performed in a natural, modified natural or HRT cycle. The day of biopsy in a natural or modified natural cycle is determined according to the LH surge or the administration of hCG, respectively, which is considered to be day zero (LH+0 or hCG+0). In both cases, the endometrial biopsy is often taken (but not restricted to) seven days later on day LH+7 or hCG+7. In an HRT cycle, the day of biopsy is determined according to the start of the P4 treatment, which again is considered to be day zero (P+0). The biopsy is then taken after five full days of P4 administration on day P+5.

The assignment of the ER status to a sample is based on the comparison of the gene expression profile of the biological sample obtained from a subject to the gene expression profile of a predictor (reference group) using a classification model. The predictor is generated from gene expression data derived from samples with known receptivity status obtained from fertile women or patients at different stages during a natural or HRT cycle. For example, samples obtained during the proliferative endometrial phase refer to a non-receptive endometrial status, whereas samples obtained prior, during or after the WOI refer to a pre-receptive endometrial status, receptive endometrial status, or post-receptive endometrial status, respectively (FIG. 4). Using the predictor as a reference, an ER status is then assigned to a sample, which can be non-receptive, pre-receptive, receptive or post-receptive.

Following the assignment of a receptivity status to a sample, a patient-specific personalized/precision embryo transfer (PET) is performed in a new cycle. An embryo (i.e., a blastocyst) is transferred on the day predicted to be receptive as of the previous cycle (FIG. 5). For example, if the gene expression profile of the biopsied sample is determined to correspond to a receptive endometrial gene expression profile, i.e., the gene expression profile of the biopsied sample is concordant with that of a normal receptive endometrium gene expression profile, embryonic transfer is scheduled in a new cycle on the day the biopsy was previously taken.

If the sample is determined to have a pre-receptive or post-receptive endometrial gene expression profile, a second biopsy can be performed to confirm the test result. For instance, if the gene expression profile of the first biopsied sample is determined to correspond to a pre-receptive endometrial gene expression profile, a second biopsy is performed one or two days later in a subsequent cycle. In contrast, if the gene expression profile of the first biopsied sample is determined to correspond to a post-receptive endometrial gene expression profile, a second biopsy is performed one or two days earlier in a subsequent cycle. If the gene expression profile of the second biopsied sample is determined to correspond to a receptive endometrial gene expression profile, a PET is scheduled and is performed on the day the second biopsy was taken in a new cycle. If the gene expression profile of the second biopsied sample is determined to correspond to a non-receptive endometrial gene expression profile, the treatment plan is re-evaluated.

The diagnosis of receptivity is valid for the type of cycle (e.g., natural cycle, modified natural cycle, HRT cycle) in which the endometrial gene expression profile is determined, and therefore embryo transfer (ET) is performed in the same type of cycle. For example, if the biopsy is taken in an HRT cycle, ET is performed in an HRT cycle, when the endometrial gene expression profile is determined to correspond to a receptive endometrial gene expression profile.

### Example 2. Endometrial Receptivity Testing Using OpenArray® Technology

### Technology Overview

The OpenArray® technology is a real-time qPCR technology that uses small arrays of the size of a microscope slide. For example, each array contains 3072 through-holes, organised in 48 sub-arrays. Each through-hole represents the site of a single real-time qPCR reaction performed in a 33 nl reaction volume. In each sub-array one sample is processed and total of four arrays can be processed simultaneously which means that 192 samples (48×4) can be analysed together. The technology is fully automated and loading of the array with sample DNA is automated and performed by a robotic system. In some embodiments of any of the methods described herein, endometrial receptivity testing can include one or more (e.g., 2, 3, 4, 5, 6, 7, 8, 9, 10) arrays.

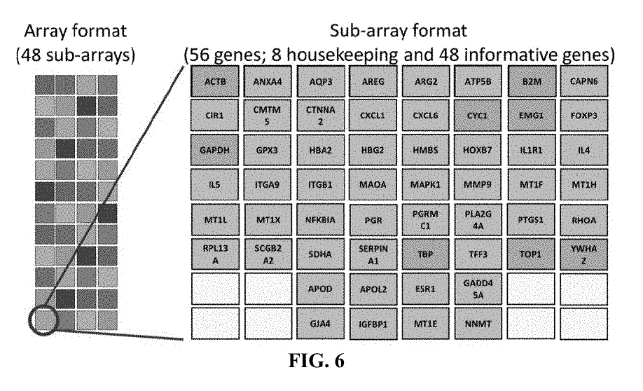

These arrays are custom-made and each through-hole is pre-spotted with one specific primer/probe set targeting a single gene (FIG. 6). A total of 56 different gene loci are assessed per sample, of which eight genes serve as potential endogenous controls for data normalisation. The remaining 48 genes correspond to the panel of genes identified to assign endometrial receptivity status. The following primer/probe pairs were selected from a wide range of partly pre-tested assays available from Thermo Fisher Scientific: Hs01060665_g1, Hs00984874_m1, Hs01105469_g1, Hs00950669_m1, Hs00982833_m1, Hs00969569_m1, Hs00187842_m1, Hs00560073_m1, Hs00799040_s1, Hs01025850_m1, Hs01093122_m1, Hs00236937_m1, Hs00605742_g1, Hs00357717_m1, Hs00362422_m1, Hs01085834_m1, Hs04420632_g1, Hs01078668_m1, Hs00361191_g1, Hs00361131_g1, Hs00609296_g1, Hs04187556_m1, Hs00991010_m1, Hs00174122_m1, Hs00174200_m1, Hs00979865_m1, Hs00559595_m1, Hs00165140_m1, Hs01046830_m1, Hs00234579_m1, Hs00744661_sH, Hs00823168_g1, Hs03654430_s1, Hs00745167_sH, Hs00355671_g1, Hs01556702_m1, Hs00998344_m1, Hs00233352_m1, Hs00377726_m1, Hs00357608_m1, Hs03043885_g1, Hs00267190_m1, Hs00188166_m1, Hs00165475_m1, Hs00427620_m1, Hs00902278_m1, Hs00243257_m1, Hs03044281_g1, Hs00155794_m1, Hs01935263_s1, Hs01046816_m1, Hs00169255_m1, Hs00704917_s1, Hs00236877_m1, Hs01938284_g1 and Hs00196287_m1. Care was taken when selecting the 56 primer/probe pairs to ensure that most primer pairs are located on two different exons and that most probes span an exon junction to avoid detection of genomic DNA. The OpenArray® technology utilises TaqMan® chemistry, which makes use of fluorescence emitted during the amplification process. The increase in template DNA during amplification is monitored at each PCR cycle by measuring the accumulated amount of fluorescence dye coming from the cleaved probes. Fluorescence signal intensities are then converted into threshold cycle (Ct) values which are assigned to each gene. Since fluorescence signal intensities are directly proportional to the amount of generated DNA amplicons, relative DNA quantities are inferred by normalisation to control genes. To generate realtive gene expression quantities an algorithm based on the delta delta threshold cycle (ΔΔCt) method (Livak and Schmittgen, Methods 2001; 25(4): 402-408) is applied.

To assign sample receptivity status, a classification test is applied to compare sample-specific gene expression profiles to reference group gene expression profiles with known receptivity status (e.g., receptive endometrial status, non-receptive endometrial status, pre-receptive endometrial status, post-receptive endometrial status).

### RNA Extraction and Reverse Transcription

The endometrial tissue is digested, and the RNA is extracted using the RNeasy® Mini Kit (Qiagen) according to the manufacturer's recommendations. Reverse transcription is performed using the High-Capacity cDNA Reverse Transcription Kit (Thermo Fisher Scientific) following the manufacturer's instructions with slight modifications. In brief, a mastermix is prepared containing 1.0 μl of 10×RT Buffer, 1 μl of 10×RT Random Primers, 0.4 μl of 25×dNTP Mix, 0.5 μl of MultiScribe™ Reverse Transcriptase and 2.1 μl of nuclease-free water per sample. 5 μl of mastermix is combined with 5 μl of RNA sample and incubated in a thermal cycler as recommended by the supplier.

### Multiplex Pre-Amplification

To enrich the 56 genes of interested a multiplex pre-amplification is performed. A mastermix is prepared consisting of 8.3 μl of TaqMan® PreAmp Master Mix (Thermo Fisher Scientific), 4.2 μl of TaqMan® Custom PreAmp Pool and 3.36 μl of nuclease-free water per sample. 15.86 μl of the mastermix is combined with 0.84 μl of cDNA and incubated in a thermal cycler according to the following settings: 1 cycle of 95.00° C. for 10 minutes, 16 cycles of 95.00° C. for 15 seconds and 60.00° C. for 4 minutes and 1 cycle of 99.00° C. for 10 minutes.

Quantitative Real-Time PCR Pre-amplified samples are diluted in nuclease-free water (1:10 dilution) and then combined with TaqMan® OpenArray Real-Time PCR Master Mix (Thermo Fisher Scientific) at a ratio of 1:4. Mixtures are then transferred to a 384 well-plate and loaded onto the arrays by a robotic system (QuantStudio™ 12K Flex OpenArray® AccuFill System, Thermo Fisher Scientific). Real-time qPCR reactions are performed on a QuantStudio™ 12K Flex Real-Time PCR System (Thermo Fisher Scientific).

### Example 3. Identification of a Panel of 48 Endometrial Receptivity Genes

A literature review was performed in order to identify previously studied genes related to endometrial receptivity, embryo implantation, and maternal immune response. Following this review, a total of 184 genes were identified that may serve as potential markers for endometrial receptivity using the OpenArray® technology (Table 3). Non-limiting examples of control/housekeeping genes used for data normalisation are shown in Table 4.

TABLE 3
List of 184 genes identified during the literature review

| | | NCBI Accession |
|---|---|---|
| Gene Symbol | Gene Name | No. |
| ABCC3 | ATP binding cassette subfamily | NM_003786.3 |
| | C member 3 | |
| ACTA1 | Actin, alpha 1, skeletal muscle | NM_001100.3 |
| ALDH1A3 | Aldehyde dehydrogenase family 1 | NM_000693.3 |
| | member A3 | |
| AMIGO2 | Adhesion molecule with Ig like | NM_001143668.1 |
| | domain 2 | |
| ANGPTL1 | Angiopoietin-related protein 1 | NM_004673.3 |
| ANXA2 | Annexin A2 | NM_001002857.1 |
| ANXA4 | Annexin A4 | NM_001153.4 |
| APOD | Apolipoprotein D | NM_001647.3 |
| APOE | Apolipoprotein E | NM_001302688.1 |
| APOL2 | Apolipoprotein L2 | NM_030882.3 |
| AQP3 | Aquaporin-3 (Gill blood group) | NM_004925.4 |
| AREG | Amphiregulin | NM_001657.3 |
| ARG2 | Arginase-2 | NM_001172.3 |
| ARHGDIA | Rho GDP-dissociation inhibitor | NM_001185077.2 |
| | alpha | |
| ATP5F1B | ATP synthase F1 subunit beta | NM_001686.3 |
| BTC | Betacellulin | NM_001729.3 |
| C2CD4B | C2 calcium-dependent domain- | NM_001007595.2 |
| | containing 4B | |
| C4BPA | Complement component 4 binding | NM_000715.3 |
| | protein alpha | |
| CALCA | Calcitonin-related polypeptide | NM_001033953.2 |
| | alpha | |
| CALR | Calreticulin | NM_004343.3 |
| CAPN6 | Calpain-6 | NM_014289.3 |
| CATSPERB | Cation channel sperm-associated | NM_024764.3 |
| | protein subunit beta | |
| CCL2 | C-C motif chemokine ligand 2 | NM_002982.3 |
| CCR7 | C-C motif chemokine receptor | NM_001838.3 |
| | type 7 | |
| CD55 | CD55 molecule (Cromer blood | NM_000574.4 |
| | group) | |
| CDA | Cytidine deaminase | NM_001785.2 |
| CDH1 | Cadherin-1 | NM_004360.4 |
| CIR1 | Corepressor interacting with | NM_004882.3 |
| | RBPJ, 1 | |
| CLDN4 | Claudin-4 | NM_001305.4 |
| CLIC1 | Chloride intracellular channel | NM_001287593.1 |
| | protein 1 | |
| CLU | Clusterin | NM_001831.3 |
| CMTM5 | CKLF-like MARVEL | NM_138460.2 |
| | transmembrane domain-containing | |
| | protein 5 | |
| COL16A1 | Collagen type XVI alpha 1 chain | NM_001856.3 |
| CRHR2 | Corticotropin-releasing hormone | NM_001883.4 |
| | receptor 2 | |
| CRISP3 | Cysteine-rich secretory protein 3 | NM_006061.3 |
| CSF1 | Colony Stimulating Factor 1 | NM_000757.5 |
| CSF3 | Colony-stimulating factor 3 | NM_000759.3 |
| CSRP2 | Cysteine and glycine-rich protein 2 | NM_001321.2 |
| CTNNA2 | Catenin alpha 2 | NM_001282597.2 |
| CXCL1 | C-X-C motif chemokine ligand 1 | NM_001511.3 |
| CXCL14 | C-X-C motif chemokine ligand 14 | NM_004887.4 |
| CXCL6 | C-X-C motif chemokine ligand 6 | NM_002993.3 |
| DEFB1 | Defensin beta 1 | NM_005218.3 |
| DEPP1 | DEPP, autophagy regulator | NM_007021.3 |
| DKK1 | Dickkopf WNT signaling pathway | NM_012242.3 |
| | inhibitor 1 | |
| EGF | Epidermal Growth Factor | NM_001963.5 |
| EPHB3 | EPH receptor B3 | NM_004443.3 |
| EREG | epiregulin | NM_001432.2 |
| ESR1 | Estrogen receptor 1 | NM_000125.3 |
| ESR2 | Estrogen Receptor 2 | NM_001437.2 |
| EZR | Ezrin | NM_003379.4 |
| FAM3B | Family with sequence similarity | NM_058186.3 |
| | 3 member B | |
| FAM3D | Family with sequence similarity | NM_138805.2 |
| | 3 member D | |
| FASLG | Fas ligand | NM_000639.2 |
| FGF7 | Fibroblast growth factor 7 | NM_002009.3 |
| FOXO1 | Forkhead box O1 | NM_002015.3 |
| FOXP3 | Forkhead box P3 | NM_014009.3 |
| FUT4 | fucosyltransferase 4 | NM_002033.3 |
| FZD5 | Frizzled class receptor 5 | NM_003468.3 |
| GABARAPL1 | Gamma type A receptor | NM_031412.2 |
| | associated protein like 1 | |
| GADD45A | Growth arrest and DNA damage- | NM_001924.3 |
| | inducible alpha | |
| GAST | Gastrin | NM_000805.4 |
| GDF15 | Growth differentiation factor 15 | NM_004864.3 |
| GJA4 | Gap junction protein alpha 4 | NM_002060.2 |
| GNLY | Granulysin | NM_001302758.1 |
| GPX3 | Glutathione peroxidase 3 | NM_002084.4 |
| HBA1 | Hemoglobin subunit alpha | NM_000558.4 |
| HBEGF | Heparin Binding-EGF like | NM_001945.2 |
| | growth factor | |
| HBG1 | Hemoglobin subunit gamma-1 | NM_000559.2 |
| HMBS | Hydroxymethylbilane synthase | NM_000190.3 |
| HOXA10 | Homeobox A10 | NM_018951.3 |
| HOXA11 | Homeobox A11 | NM_005523.5 |
| HOXB7 | Homeobox B7 | NM_004502.3 |
| HPRT1 | Hypoxanthine | NM_000194.2 |
| | phosphoribosyltransferase 1 | |
| HPSE | Heparanase | NM_006665.5 |
| ICAM1 | Intracellular adhesion | NM_000201.2 |
| | molecule 1 | |
| ID4 | Inhibitor of DNA binding, HLH | NM_001546.3 |
| | protein | |
| IDH1 | Isocitrate dehydrogenase | NM_005896.3 |
| | (NADP(+)) 1, cytosolic | |
| IER3 | Immediate early response 3 | NM_003897.3 |
| IFNG | Interferon gamma | NM_000619.2 |
| IGFBP1 | Insulin-like growth factor- | NM_000596.3 |
| | binding protein 1 | |
| IGFBP3 | Insulin-like growth factor- | NM_001013398.1 |
| | binding protein 3 | |
| IL10 | Interleukin 10 | NM_000572.2 |
| IL11 | Interleukin 11 | NM_000641.3 |
| IL15 | Interleukin-15 | NM_000585.4 |
| IL18 | Interleukin-18 | NM_001562.3 |
| IL1B | Interleukin 1 Beta | NM_000576.2 |
| IL1R1 | Interleukin-1 Receptor type 1 | NM_001288706.1 |
| IL2 | Interleukin 2 | NM_000586.3 |
| IL21 | Interleukin-21 | NM_021803.3 |
| IL4 | Interleukin 4 | NM_000589.3 |
| IL5 | Interleukin 5 | NM_000879.2 |
| IL6 | Interleukin 6 | NM_000600.4 |
| IL8 | Interleukin 8 | NM_000584.3 |
| ITGA2 | Integrin alpha-2 | NM_002203.3 |
| ITGA8 | Integrin alpha-8 | NM_003638.2 |
| ITGA9 | Integrin subunit alpha-9 | NM_002207.2 |
| ITGAV | Integrin subunit alpha V | NM_002210.4 |
| ITGB1 | Integrin beta-1 | NM_002211.3 |
| ITGB3 | Integrin beta-3 | NM_000212.2 |
| KCNG1 | Potassium voltage-gated | NM_002237.3 |
| | channel subfamily G member 1 | |
| LCP1 | Plastin-2 | NM_002298.4 |
| LEP | leptin | NM_000230.2 |
| LIF | LIF, interleukin 6 family | NM_002309.4 |
| | cytokine | |
| LIFR | LIF Receptor alpha | NM_001127671.1 |
| LPAR3 | Lysophosphatidic acid receptor 3 | NM_012152.2 |
| LRPPRC | Leucine-rich pentaricopeptide | NM_133259.3 |
| | repeat containing | |
| LRRC17 | Leucine-rich repeat-containing 17 | NM_001031692.2 |
| LYPD3 | Ly6/PLAUR domain-containing 3 | NM_014400.2 |
| MAOA | Monoamine oxidase A | NM_000240.3 |
| MAP2K1 | mitogen-activated protein | NM_002755.3 |
| | kinase kinase 1 | |
| MAP3K5 | Mitogen-activated protein | NM_005923.3 |
| | kinase kinase kinase 5 | |
| MAPK1 | Mitogen-activated protein | NM_002745.4 |
| | kinase 1 | |
| MAPK3 | Mitogen-activated protein | NM_002746.2 |
| | kinase 3 | |
| MAPK8 | Mitogen-activated protein | NM_001278547.1 |
| | kinase 8 | |
| MFAP5 | Microfibrillar-associated | NM_003480.3 |
| | protein 5 | |
| MMP10 | Matrix metallopeptidase 10 | NM_002425.2 |
| MMP2 | Matrix Metallopeptidase 2 | NM_004530.5 |
| MMP26 | Matrix metallopeptidase 26 | NM_021801.4 |
| MMP8 | Matrix metallopeptidase 8 | NM_002424.2 |
| MMP9 | Matrix Metallopeptidase 9 | NM_004994.2 |
| MT1E | Metallothionein-1E | NM_175617.3 |
| MT1F | Metallothionein-1F | NM_005949.3 |
| MT1G | Metallothionein-1G | NM_001301267.1 |
| MT1H | Metallothionein-1H | NM_005951.2 |
| MT1L | Metallothionein-1L | NR_001447.2 |
| MT1X | Metallothionein-1X | NM_005952.3 |
| MT2A | Metallothionein-2 | NM_005953.4 |
| MUC1 | Mucin 1, cell surface | NM_002456.5 |
| | associated | |
| MUC16 | Mucin-16, cell surface | NM_024690.2 |
| | associated | |
| MUC4 | Mucin-4, cell surface | NM_018406.6 |
| | associated | |
| MUC5B | Mucin-5B, oligomeric mucus/ | NM_002458.2 |
| | gel-forming | |
| NFKB1 | Nuclear factor kappa B | NM_003998.3 |
| | subunit | |
| NFKBIA | NF-kappa-B inhibitor alpha | NM_020529.2 |
| NFKBIE | Necrosis-Factor-kappa-B | NM_004556.2 |
| | inhibitor epsilon | |
| NNMT | Nicotinamide N- | NM_006169.2 |
| | methyltransferase | |
| OPRK1 | opioid receptor kappa 1 | NM_000912.4 |
| PAEP | Progestogen associated | NM_001018049.2 |
| | endometrial protein | |
| PGR | Progesterone Receptor | NM_000926.4 |
| PGRMC1 | progesterone receptor | NM_006667.4 |
| | membrane component 1 | |
| PLA2G16 | HRAS-like suppressor 3 | NM_007069.3 |
| PLA2G4A | phospholipase A2 group IVA | NM_024420.2 |
| PPP2R2C | Protein phosphatase 2 | NM_020416.3 |
| | regulatory subunit Bgamma | |
| PRDX1 | Peroxiredoxin 1 | NM_002574.3 |
| PRDX2 | Peroxiredoxin 2 | NM_005809.5 |
| PRKCG | Protein kinase C gamma type | NM_001316329.1 |
| PROK1 | Prokineticin-1 | NM_032414.2 |
| PTGER3 | Prostaglandin E2 receptor EP3 | NM_001126044.1 |
| | subtype | |
| PTGFR | Prostaglandin F2-alpha | NM_000959.3 |
| | receptor | |
| PTGS1 | Prostaglandin-endoperoxidase | NM_000962.3 |
| | synthase 1 | |
| PTGS2 | Prostaglandin-endoperoxidase | NM_000963.3 |
| | synthase 2 | |
| PTPRZ1 | Protein-tyrosine phosphatase, | NM_002851.2 |
| | receptor type Z1 | |
| RAC1 | Rac family small GTPase 1 | NM_018890.3 |
| RACGAP1 | Rac GTPase-activating protein1 | NM_013277.4 |
| RHOA | Ras homolog family member A | NM_001664.3 |
| RPL13A | ribosomal protein L13a | NM_012423.3 |
| S100A1 | S100 calcium binding protein A1 | NM_006271.1 |
| S100A10 | S100 calcium binding protein A10 | NM_002966.2 |
| S100A2 | S100 calcium binding protein A2 | NM_005978.3 |
| S100P | S100 calcium binding protein P | NM_005980.2 |
| SCGB2A2 | Secretoglobin family 2A member 2 | NM_002411.3 |
| SCGB3A1 | Secretoglobin family 3A member 1 | NM_052863.2 |
| SDHA | Succinate dehydrogenase complex | NM_004168.3 |
| | flavoprotein subunit A | |
| SELL | Selectin L | NM_000655.4 |
| SERPINA1 | Serpin family A member 1 | NM_000295.4 |
| SERPING1 | Serpin family G member 1 | NM_000062.2 |
| SGK1 | Serum/glucocorticoid regulated | NM_005627.3 |
| | kinase 1 | |
| SLPI | Secretory leukocyte peptidase | NM_003064.3 |
| | inhibitor | |
| SOD2 | Superoxide dismutase 2 | NM_000636.3 |
| SPDEF | SAM pointed domain-containing | NM_012391.2 |
| | ETS transcription factor | |
| SPP1 | Secreted phosphoprotein 1 | NM_001251830.1 |
| STAT3 | Signal transducer and activator | NM_139276.2 |
| | of transcription 3 | |
| STC1 | Stanniocalcin-1 | NM_003155.2 |
| STMN1 | Stathmin 1 | NM_001145454.2 |
| TAGLN2 | Transgelin-2 | NM_001277224.1 |
| TFF3 | Trefoil factor 3 | NM_003226.3 |
| TGFB1 | Transforming Growth Factor beta 1 | NM_000660.6 |
| TNC | Tenascin C | NM_002160.3 |
| TNF | Tumor Necrosis Factor alpha | NM_000594.3 |
| TNFRSF11B | Tumor necrosis factor receptor | NM_002546.3 |
| | superfamily member 11B | |
| TSPAN8 | Tetraspanin-8 | NM_004616.2 |
| VCAM1 | Vascular cell adhesion molecule 1 | NM_001078.3 |
| VEGFA | Vascular Endothelial Growth | NM_001025366.2 |
| | Factor A | |
| WISP2 | WNT1-inducible-signaling | NM_001323370.1 |
| | pathway protein 2 | |

TABLE 4
List of control genes

| Gene Symbol | Gene Name |
|---|---|
| ACTB | Actin beta |
| B2M | Beta-2-microglobulin |
| CYC1 | Cytochrome C1 |
| EMG1 | EMG1, N1-specific pseudouridine methyltransferase |
| GAPDH | Glyceraldehyde-3 phosphate dehydrogenase |
| TBP | TATA-box binding protein |
| TOP1 | DNA topoisomerase I |
| YWHAZ | Tyrosine 3-monooxygenase/tryptophan 5- |
| | monooxygenase activation protein zeta |

To determine whether the expression of the 184 genes was different prior to and during the expected window of implantation (WOI), real-time qPCR was performed on a large number of endometrial samples obtained from donors two and seven days (LH+2 and LH+7) following the LH surge. Of the 184 selected genes, 88 genes were found to be significantly differentially expressed between the two groups (Table 5).

In order to reduce the number of genes and to refine the gene expression profiles into clinically relevant groups (e.g., non-receptive endometrial status, pre-receptive endometrial status, receptive endometrial status and post-receptive endometrial status), samples with known receptivity status obtained from a large number of fertile donors and patients undergoing IVF treatment were analysed. Based on these results a total of 48 genes (Table 5; bold) were identified. This panel of 48 genes allows prediction of the endometrial receptivity status in biological samples and primer/probe sets targeting these 48 genes are spotted on the arrays used for endometrial receptivity analysis using the OpenArray® Technology.

TABLE 5
List of significantly differentially expressed genes

Gene Symbol
AMIGO2
ANXA4
APOD
APOE
APOL2
AQP3
AREG
ARG2
ATP5F1B
C4BPA
CAPN6
CD55
CDA
CIR1
CLU
CMTM5
CRISP3
CSRP2
CTNNA2
CXCL1
CXCL14
CXCL6
DEFB1
DEPP1
DKK1
ESR1
FOXP3
GABARAPL1
GADD45A
GAST
GJA4
GNLY
GPX3
HBA1
HBG1
HMBS
HOXB7
HPSE
ID4
IDH1
IFNG
IGFBP1
IL1R1
IL2
IL4
IL5
IL6
ITGA2
ITGA9
ITGB1
MAOA
MAPK1
MFAP5
MMP10
MMP26
MMP9
MT1E
MT1F
MT1G
MT1H
MT1L
MT1X
MUC16
NFKBIA
NNMT
PAEP
PGR
PGRMC1
PLA2G4A
PPP2R2C
PTGS1
RAC1
RACGAP1
RHOA
RPL13A
S100A1
S100P
SCGB2A2
SDHA
SERPINA1
SOD2
SPDEF
SPP1
STC1
TFF3
TNC
TNFRSF11B
TSPAN8
Genes in bold are used for prediction of the endometrial receptivity status.

### Example 4. Determination of Endometrial Receptivity Status in Clinical Setting

A pre-clinical validation study is performed to ensure accuracy of the described test for endometrial receptivity assessment. The validation study involves analysis of a large number of endometrial samples obtained from patients undergoing IVF treatment. All samples are tested with a highly-validated commercially available test (ERA; Igenomix) as well as the described test which utilises the OpenArray® technology for real-time qPCR. To establish test accuracy, concordance between the two tests is established.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of predicting endometrial receptivity for embryonic implantation in a human subject, the method comprising:
(a) providing a first biological sample obtained from a human subject at a first time point within a menstrual cycle;
(b) determining the gene expression profile of a panel of genes in the first biological sample,
wherein the panel of genes comprises two or more genes selected from the group consisting of: Annexin A4 (ANXA4), Apolipoprotein D (APOD), Apolipoprotein L2 (APOL2), Aquaporin-3 (AQP3), Amphiregulin (AREG), Arginase-2 (ARG2), ATP synthase F1 subunit beta (ATP5F1B), Calpain-6 (CAPN6), Corepressor interacting with RBPJ (CIR1), CKLF-like MARVEL transmembrane domain-containing protein 5 (CMTM5), Catenin alpha 2 (CTNNA2), C-X-C motif chemokine ligand 1 (CXCL1), C-X-C motif chemokine ligand 6 (CXCL6), Estrogen receptor 1 (ESR1), Forkhead box P3 (FOXP3), Growth arrest and DNA damage-inducible alpha (GADD45A), Gap junction protein alpha 4 (GJA4), Glutathione peroxidase 3 (GPX3), Hemoglobin subunit alpha (HBA1), Hemoglobin subunit gamma-1 (HBG1), Hydroxymethylbilane synthase (HMBS), Homeobox B7 (HOXB7), Insulin-like growth factor-binding protein 1 (IGFBP1), Interleukin-1 Receptor type 1 (IL1R1), Interleukin-4 (IL4), Interleukin-5 (IL5), Integrin subunit alpha-9 (ITGA9), Integrin beta-1 (ITGB1), Monoamine oxidase A (MAOA), Mitogen-activated protein kinase 1 (MAPK1), Matrix Metallopeptidase 9 (MMP9), Metallothionein-1E (MT1E), Metallothionein-1F (MT1F), Metallothionein-1H (MT1H), Metallothionein-1L (MT1L), Metallothionein-1× (MT1X), NF-kappa-B inhibitor alpha (NFKBIA), Nicotinamide N-methyltransferase (NMMT), Progesterone Receptor (PGR), progesterone receptor membrane component 1 (PGRMC1), phospholipase A2 group IVA (PLA2G4A), Prostaglandin-endoperoxidase synthase 1 (PTGS1), Ras homolog family member A (RHOA), ribosomal protein L13a (RPL13A), Secretoglobin family 2A member 2 (SCGB2A2), Succinate dehydrogenase complex flavoprotein subunit A (SDHA), Serpin family A member 1 (SERPINA1), and Trefoil factor 3 (TFF3) using quantitative real-time polymerase chain reaction (real-time qPCR) analysis; and
(c) identifying the human subject as having:
(i) a receptive endometrial status, when the determined gene expression profile corresponds to a gene expression profile of the panel of genes of a receptive endometrial receptivity reference group,
(ii) a non-receptive endometrial status, when the determined gene expression profile corresponds to a gene expression profile of the panel of genes of a non-receptive endometrial receptivity reference group,
(iii) a pre-receptive endometrial status, when the determined gene expression profile corresponds to a gene expression profile of the panel of genes of a pre-receptive endometrial receptivity reference group, or
(iv) a post-receptive endometrial status, when the determined gene expression profile corresponds to a gene expression profile of the panel of genes of a post-receptive endometrial receptivity reference group.

2. The method of claim 1, wherein the first biological sample is an endometrial biopsy obtained from the uterine fundus.

3. The method of claim 1, wherein the human subject has previously undergone assisted reproductive treatment, and the first time point is seven days after a luteinizing hormone surge.

4. The method of claim 1, wherein the human subject has previously undergone assisted reproductive treatment, and the first time point is seven days after administration of human chorionic gonadotropin (hCG).

5. The method of claim 3, wherein the human subject has previously undergone hormone replacement therapy cycles, and the first time point is five days after progesterone administration.

6. The method of claim 1, further comprising after identifying the human subject as having a receptive endometrial status, (d) transferring a pre-implantation embryo into the identified human subject.

7. The method of claim 1, further comprising after identifying the human subject as having a non-receptive endometrial status, a pre-receptive endometrial status, or a post-receptive endometrial status, (d) obtaining a second biological sample from the human subject at a second time point and repeating steps (b) and (c) on the second biological sample.

8. The method of claim 7, further comprising after identifying the human subject as having a receptive endometrial status, (e) transferring a pre-implantation embryo into the identified human subject.

9. The method of claim 7, wherein the second biological sample is an endometrial biopsy obtained from the uterine fundus.

10. The method of claim 7, wherein the human subject is identified as having a post-receptive endometrial status, and the second biological sample is obtained from the human subject in another menstrual cycle, one or two days before the first biological sample was obtained from the human subject in the previous menstrual cycle.

11. The method of claim 7, wherein the human subject is identified as having a pre-receptive endometrial status, and the second biological sample is obtained from the human subject in another menstrual cycle, one or two days after the first biological sample was obtained from the human subject in the previous menstrual cycle.

12. The method of claim 7, wherein the human subject is identified as having a non-receptive endometrial status, further comprising instructing a healthcare professional to select a treatment plan for the identified human subject.

13. The method of claim 7, wherein the human subject is identified as having a non-receptive endometrial status, further comprising selecting a treatment plan for the identified human subject.

14. The method of claim 12, wherein the treatment plan comprises a hormone replacement therapy cycle.

15. The method of claim 1, wherein the human subject has a history of miscarriage(s) or stillbirth(s), and/or a history of fertility issues.

16. The method of claim 1, wherein the human subject has had one or more cycles of in vitro fertilization (IVF).

17. The method of claim 1, wherein the human subject has previously not had IVF.

18. The method of claim 1, wherein the determining step occurs on a chip, an array, a multi-well plate, or a tube.

19. The method of claim 18, wherein the determining step occurs in an array comprising through-holes pre-spotted with primer/probe pairs.

20. The method of claim 1, wherein the determining step of each gene within the panel of genes is performed in a reaction volume of 0.005 μL to 100 μL.

21. The method of claim 1, wherein the determining step of each gene within the panel of genes is performed in a reaction volume of 0.02 μL to 0.05 μL.

22. The method of claim 1, wherein the determining step is performed using a computer-assisted algorithm.

23. The method of claim 1, further comprising modifying the human subject's clinical record to identify the human subject as having a receptive endometrial status, as having a non-receptive endometrial status, as having a pre-receptive endometrial status, or as having a post-receptive endometrial status.

24. The method of claim 23, wherein the clinical record is stored in a computer readable medium.

25. A kit comprising: reagents suitable for determining an endometrial gene expression profile of a panel of genes in a biological sample obtained from a human subject, wherein the panel of genes comprises two or more genes selected from the group consisting of: ANXA4, APOD, APOL2, AQP3, AREG, ARG2, ATP5F1B, CAPN6, CIR1, CMTM5, CTNNA2, CXCL1, CXCL6, ESR1, FOXP3, GADD45A, GJA4, GPX3, HBA1, HBG1, HMBS, HOXB7, IGFBP1, IL1R1, IL4, IL5, ITGA9, ITGB1, MAOA, MAPK1, MMP9, MT1E, MT1F, MT1H, MT1L, MT1X, NFKBIA, NMMT, PGR, PGRMC1, PLA2G4A, PTGS1, RHOA, RPL13A, SCGB2A2, SDHA, SERPINA1 and TFF3.

26. The kit of claim 25, wherein the biological sample is an endometrial biopsy obtained from the uterine fundus.

27. The kit of claim 25, wherein the reagents are suitable for performing real-time qPCR.

28. The kit of claim 25, further comprising reagents suitable for performing reverse transcription and multiplex pre-amplification.

29. The kit of claim 25, further comprising a chip, an array, a multi-well plate or a tube.

30. The kit of claim 29, wherein the array comprises through-holes pre-spotted with primer/probe pairs.

31. The kit of claim 25, further comprising instructions for use of the kit according to claim 1.
